(19) United States
(12) Patent Application Publication
(10) Pub. No.: US 2021/0375437 A1
(43) Pub. Date: Dec. 2, 2021

(54) SYSTEMS AND METHODS FOR DISCHARGE EVALUATION TRIAGE

(71) Applicant: Radial Analytics, Inc., Concord, MA (US)

(72) Inventors: Anant Vasudevan, Cambridge, MA (US); Eric H. Weiss, Acton, MA (US); Michael Rossi, Marlborough, MA (US); Thaddeus R. F. Fulford-Jones, Somerville, MA (US)

(21) Appl. No.: 16/889,396

(22) Filed: Jun. 1, 2020

(51) Int. Cl. G16H 40/20; G16H 50/70; G16H 10/60; A61B 5/00

(52) U.S. Cl. CPC G16H 40/20; A61B 5/7435; G16H 10/60; G16H 50/70

(57) ABSTRACT

Various aspects of the subject technology relates to systems and methods for transition of care decision intervention using machine learning. A system may be configured to receive patient data including one or more features values for a plurality of features associated with associated with one or more patients. The system may determine a first transition of care decision score for a patient by processing the patient data through a first, historical decision-derived transition of care decision model, and also determine at least a second transition of care decision score for the patient by processing the patient data through at least a first, expert recommendation-derived transition of care decision model. The system may calculate a first transition of care decision intervention priority score for the patient based on a degree of difference between the first and second transition of care decision scores for the patient.

### BACKGROUND

Medical expenditure has been growing at an unsustainable rate. To stem this, the U.S. healthcare system has begun shifting from fee-for-service to value-based care, i.e., healthcare reimbursements being primarily contingent on quality of care rather than quantity of services delivered. As part of this, providers, such as hospitals and health systems, and payers, such as health insurers and the federal government (through Medicare), share in the cost of inpatient, outpatient, and post-acute care. Post-acute care, also known as “after-hospital care” or “rehabilitation,” applies primarily to patients over 65 years who need additional care to fully recover after a hospitalization. Post-acute care also happens to be a key driver of unnecessary expenditure (amounting to about $12B/year). Thus, in view of the recent shift in healthcare reimbursements for cost of care primarily being contingent on the quality of care, and the enormous healthcare expenditure associated with such care, there is an increasing need for well-informed, high quality and cost-effective decisions around a patient's optimal health care services, care providers, and/or site of care (e.g., post-acute care) that is focused on transitioning the right patient to the right health care service, care provider and/or care site or facility.

### SUMMARY

According to one aspect, the disclosure relates to a computer-implemented method for transition of care decision intervention using machine learning. The method includes receiving patient data including values for a plurality of features associated with a first patient. In some implementations, features from a majority of the following feature categories are included: patient demographic data, patient clinical data, patient financial data, administrative data including patient health insurance information and claims data, patient health care utilization history, patient prior recovery data, data indicative of patient's access to physicians and clinical caregivers, patient socio-economic data, and patient behavioral health data. The method includes determining a first transition of care decision score for the first patient by processing the patient data through a first, historical decision-derived transition of care decision model and at least a second transition of care decision score for the first patient by processing the patient data through at least a first, expert recommendation-derived transition of care decision model. The method includes calculating a first transition of care decision intervention priority score for the first patient based on the degree of difference between the first and second transition of care decision scores for the first patient. The method further includes displaying on a graphical interface, data corresponding to the first transition of care decision intervention priority score for the first patient. In some implementations, the transition of care decision intervention includes revaluating or assigning additional resources to a health facility discharge decision, clinical triage decision, functional assessment, social needs assessment, and/or a care plan associated with the transition of care.

In some implementations, the method further includes receiving patient data including values for the features associated with at least one additional patient, determining transition of care decision scores for at least one additional patient; calculating a transition of care decision intervention priority score for at least one additional patient; and displaying on a graphical user interface, the data corresponding to transition of care decision intervention priority score for at least one additional patient. In some implementations, the first patient and the one additional patient may form a patient population. In some implementations, the data corresponding to the transition of care priority scores for the patients in the patient population is displayed on the graphical user interface, organized based on a ranking of the patients in the patient population according to their relative transition of care decision intervention priority scores. In some implementations, the patient population includes the patient population of a health care facility.

In some implementations, the method further includes determining at least one additional transition of care decision score for the first patient by processing the patient data through at least one additional expert recommendation-derived transition of care decision model; and calculating an aggregated value for an expert recommendation-derived transition of care decision score for the first patient by performing an aggregation function on the second transition of care decision score and the one or more additional transition of care decision scores determined by processing the patient data through the respective first and the one or more additional expert recommendation-derived transition of care decision models. In some implementations, calculating the first transition of care decision intervention priority score for the first patient is based on the degree of difference between the first transition of care decision score and the aggregated value for the expert recommendation-derived transition of care decision score.

In some implementations, the method further includes displaying on the graphical user interface at least one or more of the following information types:
(a) explanatory information underlying a transition of care decision intervention recommendation for a patient comprising at least one of:
   (i) clinical justifications for a transition of care decision intervention,
   (ii) indicators of socio-behavioral needs,
   (iii) markers of frailty and decreased mobility, and/or
   (iv) prior health care utilization and recovery history; and
(b) a personalized list of recommendations for a patient comprising at least one of:
   (i) recommended health care services, care providers, facilities, and/or agencies cross-checked with a patient's medical insurance,
   (ii) recommendations for follow-up assessments,
   (iii) recommendations for clinical interventions by future providers, and/or
   (iv) a recommended duration for at least one or more of the following: a clinical intervention, institutionalization, series of home health care provider visits, and/or hospitalization.

According to certain aspects of the present disclosure, a non-transitory computer-readable medium storing program instructions is provided, that, when executed by a processor, causes the processor to perform a method for transition of care decision intervention using machine learning. The program instructions stored on the non-transitory computer-readable medium perform the method including receiving patient data including values for a plurality of features associated with a first patient. The program instructions further perform the method including determining a first transition of care decision score for the first patient by processing the patient data through a first, historical decision-derived transition of care decision model and at least a second transition of care decision score for the first patient by processing the patient data through at least a first, expert recommendation-derived transition of care decision model. The program instructions further perform the method including calculating a first transition of care decision intervention priority score for the first patient based on the degree of difference between the first and second transition of care decision scores for the first patient. The program instructions further perform the method including displaying on a graphical interface, data corresponding to the first transition of care decision intervention priority score for the first patient.

In some implementations, the program instructions stored on the non-transitory computer-readable medium further perform the method including: receiving patient data including values for the features associated with at least one additional patient, determining transition of care decision scores for at least one additional patient; calculating a transition of care decision intervention priority score for at least one additional patient; and displaying on a graphical user interface, the data corresponding to transition of care decision intervention priority score for at least one additional patient. In some implementations, the first patient and the one additional patient may form a patient population. In some implementations, the data corresponding to the transition of care priority scores for the patients in the patient population is displayed on the graphical user interface, organized based on a ranking of the patients in the patient population according to their relative transition of care decision intervention priority scores.

In some implementations, the program instructions stored on the non-transitory computer-readable medium further perform the method including displaying on the graphical user interface at least one or more of the following information types:

(a) explanatory information underlying a transition of care decision intervention recommendation for a patient comprising at least one of:
(i) clinical justifications for a transition of care decision intervention,
(ii) indicators of socio-behavioral needs,
(iii) markers of frailty and decreased mobility, and
(iv) prior health care utilization and recovery history; and
   (b) a personalized list of recommendations for a patient comprising at least one of:
(i) recommended health care services, care providers, facilities, and/or agencies cross-checked with a patient's medical insurance,
(ii) recommendations for follow-up assessments,
(iii) recommendations for clinical interventions by future providers, and
(iv) a recommended duration for at least one or more of the following: a clinical intervention, institutionalization, series of home health care provider visits, and hospitalization.

According to certain aspects of the present disclosure, a system for transition of care decision intervention using machine learning is provided. The system includes a memory storing computer-readable instructions and a plurality of transition of care decision intervention models. The system also includes a processor configured to execute the computer-readable instructions. The instructions, when executed causes the processor to receive patient data including values for a plurality of features associated with a first patient. In some implementations, features from a majority of the following feature categories are included: patient demographic data, patient clinical data, patient financial data, administrative data including patient health insurance information and claims data, patient health care utilization history, patient prior recovery data, data indicative of patient's access to physicians and clinical caregivers, patient socio-economic data, and patient behavioral health data. The processors are further configured to determine a first transition of care decision score for the first patient by processing the patient data through a first, historical decision-derived transition of care decision model and a second transition of care decision score for the first patient by processing the patient data through at least a first, expert recommendation-derived transition of care decision model. The processors are further configured to calculate a first transition of care decision intervention priority score for the first patient based on the degree of difference between the first and second transition of care decision scores for the first patient. The processors are further configured to display on a graphical interface, data corresponding to the first transition of care decision intervention priority score for the first patient. In some implementations, the transition of care decision intervention includes revaluating or assigning additional resources to a health facility discharge decision, clinical triage decision, functional assessment, social needs assessment, and/or a care plan associated with the transition of care.

In some implementations, the memory is further configured to store computer-readable instructions, which when executed cause the processor to receive patient data including values for a plurality of features associated with at least one additional patient; determining transition of care decision scores for at least one additional patient; calculating a transition of care decision intervention priority score for at least one additional patient; and displaying on a graphical user interface, the data corresponding to transition of care decision intervention priority score for at least one additional patient. In some implementations, the first patient and at least one additional patient form a patient population. In some implementations, the data corresponding to the transition of care priority scores for the patients in the patient population is displayed on the graphical user interface, organized based on a ranking of the patients in the patient population according to their relative transition of care decision intervention priority scores. In some implementations, the patient population includes the patient population of a health care facility.

In some implementations, the memory further stores computer-readable instructions, which when executed cause the processor to determine at least one additional transition of care decision score for the first patient by processing the patient data through at least one additional expert recommendation-derived transition of care decision model; calculating an aggregated value for an expert recommendation-derived transition of care decision score for the first patient by performing an aggregation function on the second transition of care decision score and at least one additional transition of care decision score determined by processing the patient data through respective first and the at least one additional expert recommendation-derived transition of care decision models; and calculating the first transition of care decision intervention priority score for the first patient based on the degree of difference between the first transition of care decision score and the said aggregated value for the expert recommendation-derived transition of care decision score.

In some implementations, the memory further stores computer-readable instructions, which when executed cause the processor to display on the graphical user interface at least one or more of the following information types:

(a) explanatory information underlying a transition of care decision intervention recommendation for a patient comprising at least one of:
(i) clinical justifications for a transition of care decision intervention,
(ii) indicators of socio-behavioral needs,
(iii) markers of frailty and decreased mobility, and/or
(iv) prior health care utilization and recovery history; and
   (b) a personalized list of recommendations for a patient comprising at least one of:
(i) recommended health care services, care providers, facilities and/or agencies cross-checked with a patient's medical insurance,
(ii) recommendations for follow-up assessments,
(iii) recommendations for clinical interventions by future providers, and/or
(iv) a recommended duration for at least one or more of the following: a clinical intervention, institutionalization, series of home health care provider visits, and/or hospitalization.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example architecture for determining transition of care decision intervention priority scores for transition of care decision interventions using machine learning according to some implementations of the systems and methods as disclosed herein.

FIGS. 2A-2E illustrate example block diagrams of systems for determining transition of care decision intervention priority scores for transition of care decision interventions using machine learning according to some implementations of the systems and methods as disclosed herein.

FIGS. 3A and 3B are flowcharts showing a method for determining transition of care decision intervention priority scores using historical decision-derived and expert recommendation-derived transition of care decision models derived from a machine learning process according to some implementations of the systems and methods as disclosed herein.

FIGS. 4A-4C illustrate example user interfaces for displaying and interacting with transition of care decision intervention priority scores according to some implementations of the systems and methods as disclosed herein.

FIG. 5 is a block diagram of an example computing system.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Thus, while the following detailed description section may include information that describes one or more aspects of the subject technology, various embodiments described in the present disclosure may be carried out in different ways and variations, and in accordance with a desired application or implementation.

Disclosed systems and methods advantageously use algorithms and machine learning applications to create a tool to enable cost-effective and high-quality decisions around a patient's optimal health care services, care providers, and/or site of care (e.g., post-acute care) that is focused on transitioning the right patient to the right health care services, care providers, and/or care site or facility and other similar transition of care decisions.

Machine learning is an application of artificial intelligence that automates the development of an analytical model by using algorithms that iteratively learn patterns from data without explicit indication of the data patterns. Machine learning is commonly used in pattern recognition, computer vision, email filtering, and optical character recognition, and enables the construction of algorithms that can accurately learn from data to predict model target outputs thereby making data-driven predictions or decisions.

Aspects of the present disclosure relate to systems and methods that empower healthcare practitioners, providers, and clinicians to determine whether a transition of care decision intervention is necessary for a given patient. The systems and methods disclosed herein take into account at least two or more models, i.e., a baseline historical decision-derived transition of care decision model and an expert recommendation-derived transition of care decision model each of which are generated and trained during a machine learning process. In broad overview, the historical decision-derived transition of care decision model disclosed herein is trained during the machine learning process using training data that includes patient data from a relevant healthcare facility, system, or setting, and historical transition of care decisions made at that healthcare facility, system, or setting based on the respective patient data for the patients. On the other hand, the expert recommendation-derived transition of care decision model disclosed herein is trained during the machine learning process using training data that includes the same or different patient data from the same or different healthcare facility, healthcare system, or healthcare setting, and independent expert transition of care decision recommendations based on expert reviews of the relevant patient data for such patients.

The trained transition of care decision models are then utilized for processing a wide variety of received execution patient data as input and determining a respective historical decision model-derived transition of care decision score and an expert recommendation model-derived transition of care decision score for one or more patients. A transition of care decision intervention priority score is then determined based on the degree of difference between the respective transition of care decision scores. A determination is then made as to whether a transition of care decision intervention is necessary for a patient based on the patient's transition of care decision intervention priority score, and in some implementations, an intervention priority classification corresponding to the intervention priority score. The transition of care decision intervention determination is then provided to relevant healthcare practitioners, providers, and clinicians involved with a patient's transition of care decision.

The term “intervention,” as used herein, refers to interrupting the standard transition of care decision making process. Such intervention may include, but is not limited to, a reevaluation of the optimal health care services, care providers, and/or site of care (e.g., post-acute care) and/or additional functional assessments of the patient by a healthcare expert, such as a healthcare practitioner, provider, or a clinician. It should be understood that, the term “optimal,” as used herein, is intended to mean a medically preferred option given the known information, and may not necessarily be “perfectly optimal” given the uncertainties of the medical sciences and imperfect information availability. It is further understood that, as used herein, the optimal or recommended “sites” and “services” are meant to be generic sites and services, whereas the recommended “provider” and/or “facility” is meant to be a specific provider of a given service and/or a specific facility of a site type. Example services include rehabilitation, physical therapy, psychiatric counseling, palliative care, etc., whereas example providers include specific practitioners, clinicians, medical groups, physical therapy providers, etc. Example sites include rehabilitation hospitals, hospices, the patient's home, a skilled nursing facility, hospital ward type, etc. Example of facilities include specific hospitals, medical centers, hospice locations, etc. In some implementations, intervention may also include reevaluating or assigning additional resources to a health facility discharge decision, a clinical triage decision, functional assessment, social needs assessment, and/or a care plan associated with the transition of care. Additionally, or alternatively, the intervention may also include additional social evaluation of the patient by a social worker. Additionally, or alternatively, the intervention may also include additional functional assessment of the patient by a suitable health care provider. As discussed in details in the following sections, the systems and methods described herein are utilized to determine whether a transition of care decision intervention is necessary for one or more patients based on the differences in the transition of care decision scores determined by the two trained models. The systems and methods described herein, however, do not assume that one of the models, i.e., the historical decision-derived transition of care decision model or the expert recommendation-derived transition of care decision model, is more accurate in determining a transition of care decision score or a transition of care decision intervention than the other. Thus the outputs of the models disclosed herein may not necessarily be used to determine an actual transition of care decision for any given patient.

The systems and methods disclosed herein primarily relate to transition of care decisions regarding discharging a patient from a hospital facility to home or homecare rather than a skilled nursing facility (SNF). The systems and methods disclosed herein can be further used for other transition of care decisions, i.e., discharge decisions regarding optimal post-transition health care services, care providers, and/or a site of care including, but not limited to, discharge from emergency department (ED) to inpatient hospitalization, discharge from inpatient hospitalization to a variety of post-acute care services, providers, and sites including, for example, Home Health Agencies (HHA), Skilled Nursing Facilities (SNF), Inpatient Rehabilitation Facilities (IRF), Long-Term Acute Care hospitals (LTACHs), discharge from inpatient hospitalization to hospice care, discharge from post-acute care to outpatient services, discharge from post-acute care to home or home care, and discharge from intensive care unit (ICU) to inpatient care. The systems and methods disclosed herein can also be used for other transition of care decisions, e.g., for certain patient segments (including Medicaid) and any other clinical decision spaces more broadly involving transition of care.

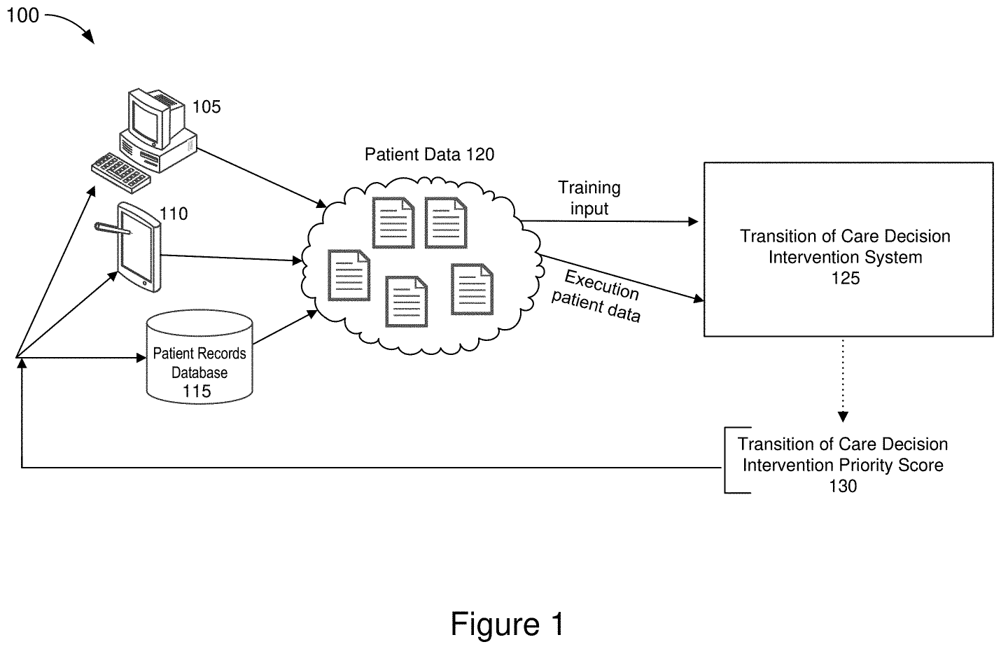

FIG. 1 illustrates an example architecture 100 for determining transition of care decision intervention priority scores for transition of care decision interventions using machine learning. The architecture 100 includes a large-format computing device 105, a small-format computing device 110, a patient records database 115, and patient data 120. The architecture 100 also includes a transition of care decision intervention system 125 that determines a transition of care decision intervention priority score 130 for one or more patients. A patient population, as used herein, refers to at least two or more of the patients in a given health care facility or health care system at a given time. Additional details of the machine learning process used herein to determine transition of care decision intervention priority scores can be found below.

As shown in FIG. 1, a large-format computing device 105 or any other fully functional computing device, such as a desktop computer or laptop computer, may transmit patient data 120 to the transition of care decision intervention system 125. Additionally, or alternatively, other computing devices, such as a small-format computing device 110 may also transmit patient data 120 to the transition of care decision intervention system 125. Small-format computing device 110 may include a tablet, smartphone, personal digital assistant (PDA), or any other computing device that may have more limited functionality compared to large-format computing devices 105. Patient data may be stored in a database, for example in a patient records database 115 to be transmitted to the transition of care decision intervention system 125. Large-format computing device 105 and small-format computing device 110 may include memory storing data and applications related to determining and displaying patient transition of care decision intervention priority scores. In some implementations, the large-format computing device 105 and the small-format computing device 110 may receive patient data input by healthcare practitioners, other computing devices, or directly from patient monitoring equipment and may transmit the patient data to a transition of care decision intervention system 125.

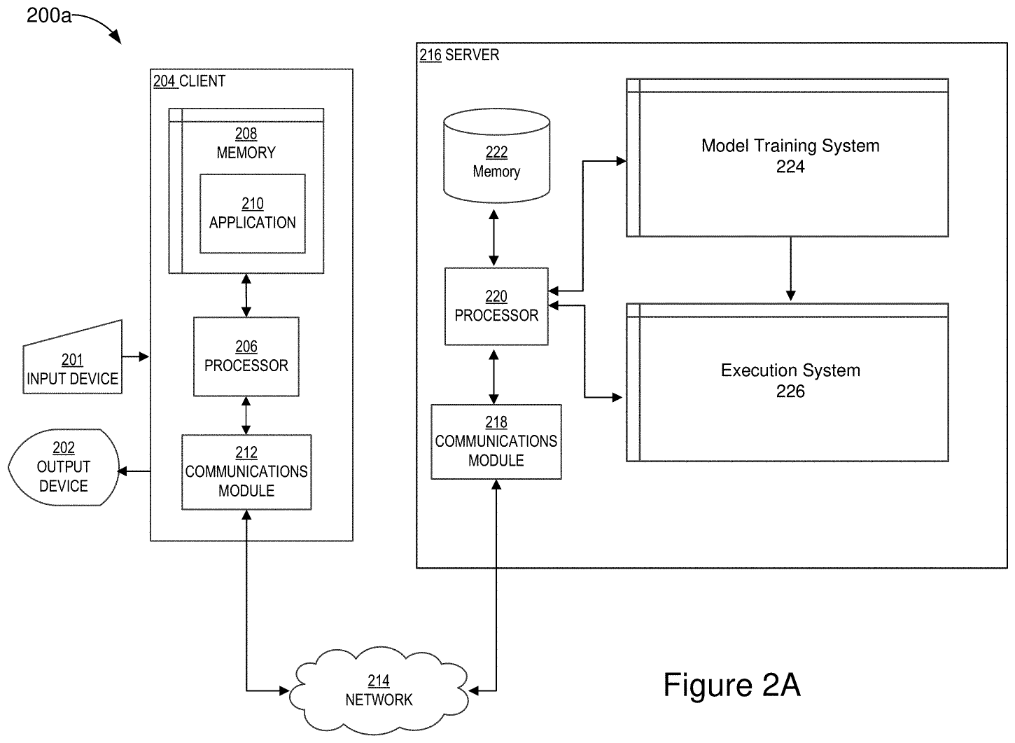

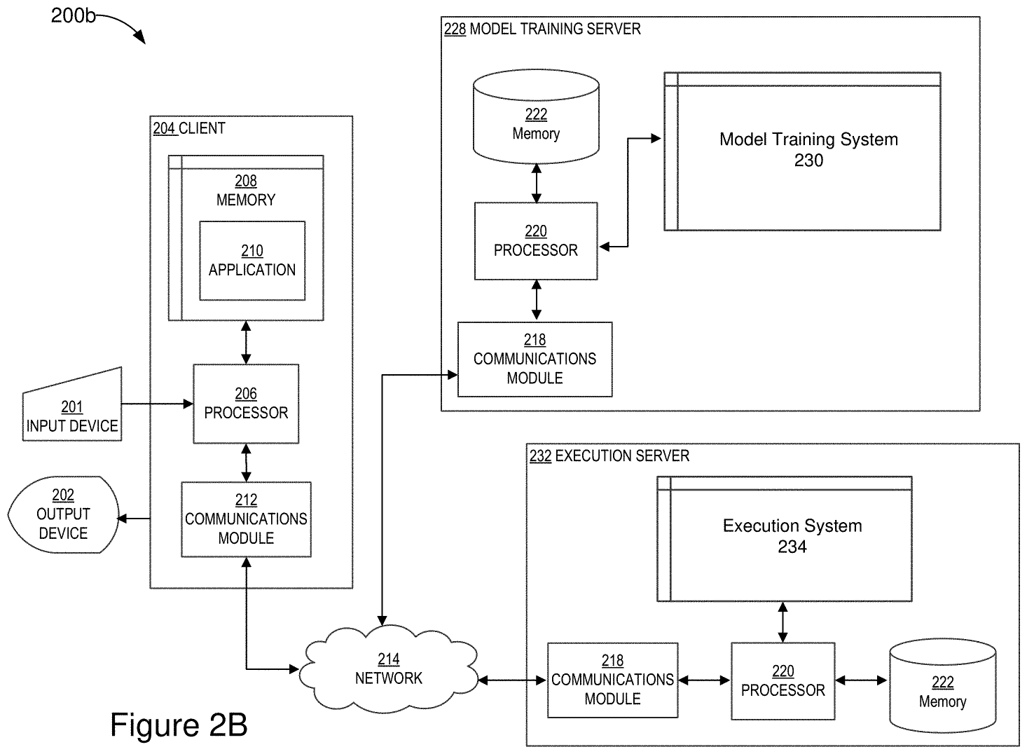

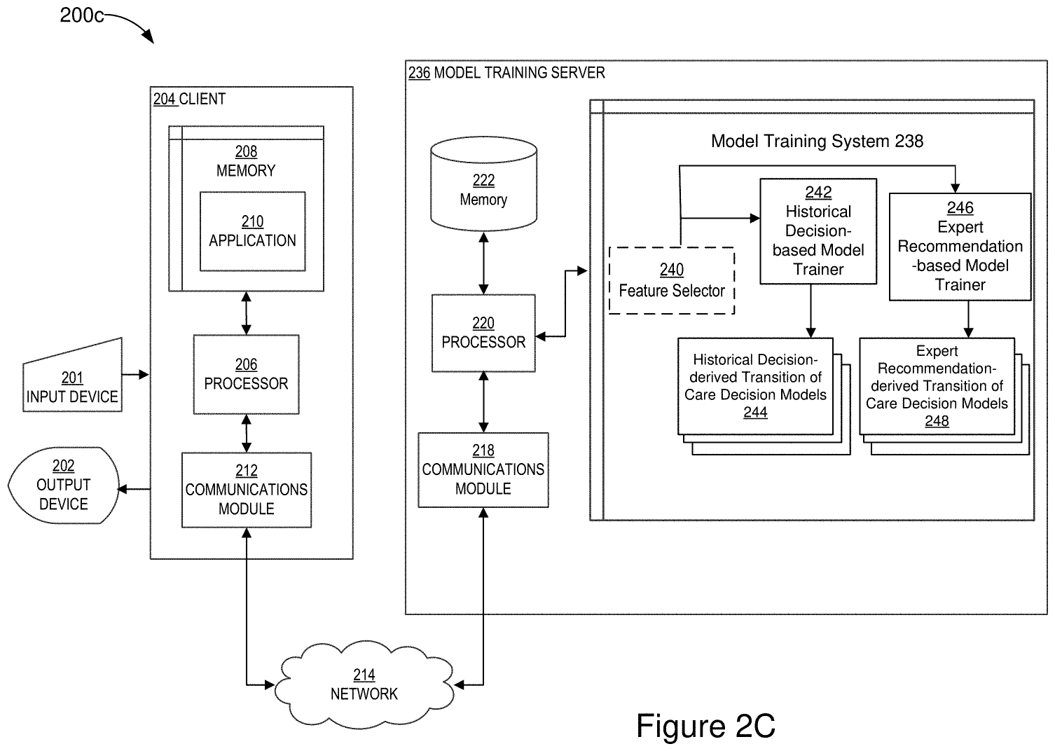

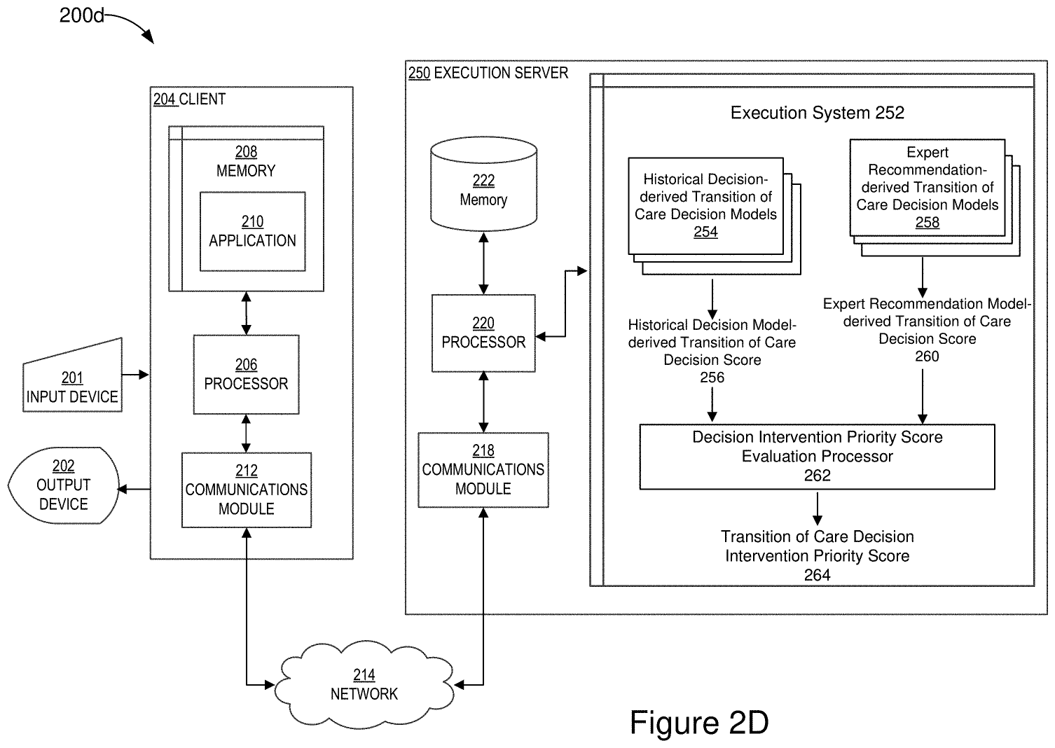

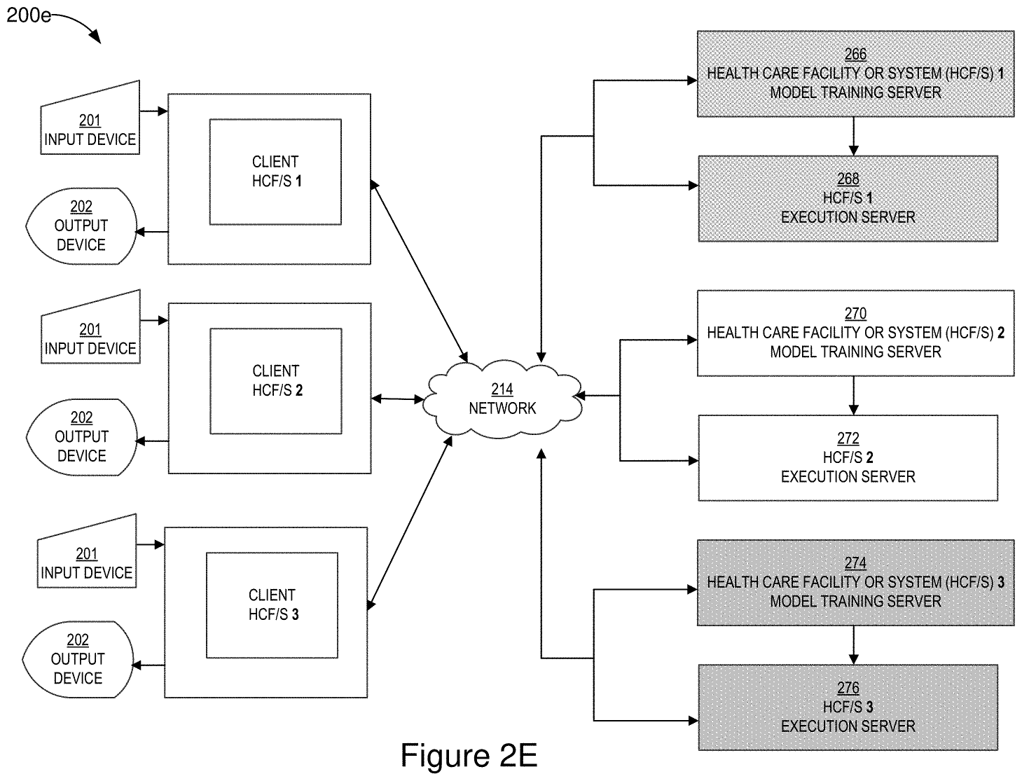

As shown in FIG. 1, patient data 120 is transmitted to a transition of care decision intervention system 125. In some implementations, the patient data 120 includes training input that is transmitted to a transition of care decision intervention system 125 for use in a machine learning process. The training input is used to train a machine learning algorithm in a machine learning process in the transition of care decision intervention system 125 in order to generate at least two or more transition of care decision models that are capable of subsequently determining transition of care decision scores and transition of care decision intervention priority scores based on a wide variety of received patient data (shown in FIG. 1 as execution patient data). In some implementations, the patient data 120 also includes execution patient data that are transmitted to the transition of care decision intervention system 125 as inputs to be processed by the generated transition of care decision models for determining a patients' transition of care decision scores and transition of care decision intervention priority scores. Thus, the execution patient data included in the patient data 120 can be processed by the generated transition of care decision models of the transition of care decision intervention system 125 in determining a transition of care decision intervention priority score for one or more patients. Additional details of the different components included in the transition of care decision intervention system 125 used herein to determine transition of care decision intervention priority score can be found below, e.g., in the description of FIGS. 2A-2E below.

The patient data 120 may include a number of standard clinical parameters or measurements, demographic data, financial data, administrative data, health care utilization history, and other inputs, collectively known as features, which are commonly collected and available in healthcare settings, or generated through processing healthcare claims or other billing data.

The clinical features of the patient data 120 may include, but are not limited to, common patient measurements, vital signs or observations, chief complaint, diagnoses and procedures, patient notes, laboratory test results, medications taken and the dosage of those medications, as well as any materials, solids, fluids entering and leaving the patient by specified routes. Examples of features related to common patient measurements, vital signs or observations may include, but are not limited to, body mass index (BMI), oxygen saturation below 92% within the past 24 hours, etc. The chief complaint feature may include a text field that includes extracted feature tokens using term frequency-inverse document frequency (TF-IDF) Natural Language Processing (NLP) such as “failure to thrive.”

Examples of features related to diagnoses and procedures may include, but are not limited to, chronic conditions, model features derived from prior diagnoses, working diagnosis-related group (DRG) (e.g., DRG=871, “Sepsis with major complication/comorbidity”), and major diagnostic category (MDC), which is a categorical roll-up of DRGs (MDC=08, “Diseases & Disorders of the Musculoskeletal System & Connective Tissue”). Examples of features related to chronic conditions include conditions derived from ICD-10 diagnoses and procedures based on the formal “Condition Categories” defined in the “CMS Chronic Conditions Data Warehouse” (https://www2.ccwdata.org/web/guest/condition-categories). Examples of “condition categories” may include, for example, mobility impairments, Alzheimer's Disease, dementia, one of multiple forms of cancer, etc.

Examples of specific model features derived from prior diagnoses may include, but are not related to the following:
“has DNR” (e.g., ICD-10 diagnosis=“Z66”)
“has cachexia or abnormal weight loss” (e.g., ICD-10 diagnosis in {“R64”, “R634”})
“has oxygen dependency” (e.g., ICD-10 diagnosis=“Z9981”)
“has failure to thrive” (e.g., ICD-10 diagnosis=“R627”)
“has malnutrition” (e.g., ICD-10 diagnosis in {“E43”, “E440”, “E441”, “E46”, “E64”, “E640”})

Examples of features related to patient notes may include, but are not limited to, physical therapy (PT) rehabilitation requirements via PT note.

Examples of features related to laboratory test results may include, but are not limited to, the following:
“has electrolyte derangement” (e.g., abnormal sodium or potassium on basic metabolic panel in the past 24 hours, past 12 hours, etc.);
“has acute renal failure” (e.g., creatinine is either 20% higher than baseline value or greater than 1.2 mg/dL in the past 24 hours, past 48 hours, past 7 days, etc.).

Examples of features related to imaging test results may include, but are not limited to, the following: “has pneumonia” (e.g., pneumonia documented on chest x-ray or CT Chest in the past 5 days); “has lung cancer” (e.g., lung cancer documented on CT scan of the lungs); etc.

Examples of features related to medications taken and dosage of those medications may include, but are not limited to, the following:
“has used an ACE inhibitor” (e.g., patient has been prescribed lisinopril, enalapril, etc. of any dose in the past 3 months, past 6 months, past 12 months, etc.);
“has been prescribed a high-intensity statin” (e.g., patient has been prescribed either atorvastatin 80 mg or rosuvastatin 40 mg in the past 3 months, past 6 months, past 12 months, etc.).

Examples of features materials, solids, fluids entering and leaving the patient by specified routes may include, but are not limited to, the following:
“has required IV fluids” (e.g. patient is receiving either sodium chloride or Lactated Ringer's infusions in the past 24 hours, past 12 hours, etc.);
“has received a flu shot.”

The demographic features of the patient data 120 may include, but are not limited to, patient age (e.g., age<60, age between 60 and 75, age>75, etc.), sex, race, ethnicity, marital status (e.g., married, unmarried, widowed, divorced, etc.), education, primary contact information, next of kin information, and home address or zip code.

Exemplary financial features of the patient data 120 include, but are not limited to, patient income, employment information (e.g., retired, employed, unemployed, etc.), and neighborhood housing characteristics, including median and mean household income, percent of owner-occupied housing, median housing value, and median gross rent.

Exemplary administrative data of the patient data 120 may include, but are not limited to, patient health insurance information (e.g., Medicare eligible, Medicaid eligible, Medicare and Medicaid (Dual eligible), etc.), and hospital unit and room information (e.g., in ICU, on a telemetry unit (cardiac unit), in surgical unit, etc.).

Some examples of the health care utilization history features of the patient data 120 include previous acute inpatient hospitalization information including, but not limited to site, duration, and purpose (e.g., “has recent acute inpatient admission (within 30 days)”; “has recent same-site acute inpatient admission (within 30 days)”, etc.). The health care utilization history features may include previous medical care provided in an emergency department (ED), in an outpatient setting, by post-acute care services, providers, and sites including Home Health Agencies (HHA), Skilled Nursing Facilities (SNF), Inpatient Rehabilitation Facilities (IRF), Long-Term Acute Care hospitals (LTACHs), and/or hospice care. Some examples of health care utilization history features are: “admitted from the ED”; “has SNF visit within the past 90 days”; “has LTAC visit within the past 90 days”; “has ongoing HHA services”; “has HHA services within the past year”; “has previously been referred to palliative care services”; and “has hospice services within the past year.”

Exemplary professional medical services include, but are not limited to, primary care and specialist visits (e.g., “has primary care visit within the past three months,” “has cardiologist visit within the past three months,” etc.) and prior use of durable medical equipment (e.g., “has walker”, “has wheelchair”, “has external oxygen”, etc.). In some embodiments, the patient data 120 may include data from previously collected claims data. It can be understood that, one or more of the exemplary features described in details in relation to the patient data 120 of FIG. 1 above, are also applicable as data or input features for execution patient data, received patient data, or any other patient input data described herein.

In some embodiments, the patient data 120 may include data from inpatient or outpatient real-time monitoring devices. In some implementations, when patient data includes data from inpatient or outpatient real-time monitoring devices, the systems and methods disclosed herein are capable of sending information related to the transition of care decision intervention back to the inpatient or outpatient real-time monitoring devices or healthcare practitioners, providers, and clinicians monitoring patients who are wearing or using such monitoring devices and/or to the patients wearing or using such monitoring devices.

As further shown in FIG. 1, architecture 100 includes a transition of care decision intervention system 125. In broad overview, the transition of care decision intervention system 125 receives patient data 120 as training input for use in a machine learning process for determining and outputting a transition of care decision intervention priority score 130 for one or more patients. The transition of care decision intervention system 125 functions in the training aspect of a machine learning process to receive patient data as training input to generate and train at least two transition of care decision models, which are then capable of determining transition of care decision scores and a transition of care decision intervention priority score, based on a wide variety of received execution patient data included in the patient data 120. Thus, in some implementations, the transition of care decision intervention system 125 additionally transmits the execution patient data included in the patient data 120 as inputs to the generated transition of care decision models and processes the execution patient data through the generated transition of care decision models in determining a transition of care decision intervention priority score for one or more patients. Additional details of the different components included in the transition of care decision intervention system 125 used herein to determine the transition of care decision intervention priority score can be found below, e.g., in the description of FIGS. 2A-2E below.

As further shown in FIG. 1, architecture 100 determines a transition of care decision intervention priority score 130 for a given patient or multiple patients. The transition of care decision intervention priority score 130 is transmitted to the large-format computing device 105, the small-format computing device 110 and/or the patient records database 115. The determined patient transition of care decision intervention priority score 130 may be output to a graphical user interface on the large-format computing device 105 and/or the small-format computing device 110. The output patient transition of care decision intervention priority score 130 may be utilized by healthcare providers to determine a transition of care decision intervention plan for one or more patients. Additionally, or alternatively, in some implementations, data corresponding to the transition of care decision scores, for one or more patients, is transmitted to the large-format computing device 105, the small-format computing device 110 and/or the patient records database 115. The data corresponding to the transition of care decision scores may be output to a graphical user interface on the large-format computing device 105 and/or the small-format computing device 110.

In some implementations, the data corresponding to the transition of care decision scores may reflect the raw historical decision model-derived transition of care decision score and the corresponding raw expert recommendation model-derived transition of care decision score for one or more patients. In some implementations the raw historical decision model-derived transition of care decision score corresponds to a probability that, as between two post-transition of care settings, a patient would historically have been transitioned to one of the two settings. For example, for a determination between whether a patient would historically have been discharged to home versus to a facility, a value of 0.0 output by a model may reflect a 100% probability that the patient would have been discharged to home and a 0% probability that the patient would have been discharged to a facility, an output of 1.0 by a model may reflect a 0% probability that the patient would have been discharged to home and a 100% probability that the patient would have been discharged to a facility, and a value of 0.5 may represent that that there is an equal probability that the patient would have been discharged to either care setting. In some implementations, the raw expert recommendation model-derived transition of care decision score corresponds to a probability that, as between two post-transition of care settings, a patient should be transitioned to one of the two settings. For example, for a determination between whether a patient should be discharged to home versus to a facility, a value of 0.0 output by a model may reflect a 100% probability that the patient should be discharged to home and a 0% probability that the patient should be discharged to a facility, an output of 1.0 by a model may reflect a 0% probability that the patient should be discharged to home and a 100% probability that the patient should be discharged to a facility, and a value of 0.5 may represent that that there is an equal probability that the patient should be discharged to either care setting. In some implementations, the raw scores output by the models may not correspond to probability values, in which case the scores for each model may be normalized to a common scale (e.g., between 0.0 and 1.0) to allow effective comparisons of model outputs.

In some implementations, in addition to, or instead of the raw model outputs, the data corresponding to the transition of care decision scores may be a transition of care decision intervention priority score. Generally, a transition of care decision intervention priority score represents a degree in difference between the outputs of one or more historical decision model-derived transition of care decision scores and one or more expert recommendation model-derived transition of care decision scores. The difference can be a simple arithmetic difference between the two scores, a percentage difference between the scores, and/or a classification of the level of difference (e.g., highest level of difference, high level of difference, medium level of difference, or low level of difference). Assuming model output scores being equal or normalized to values of between 0.0 and 1.0, in some implementations, the arithmetic difference by which the historical transition of care decision score exceeds the expert transition of care decision score may be used as the transition of care decision intervention priority score, such that a value greater than or equal to 0.8 may be classified as “highest”, a value greater than or equal to 0.6 and less than 0.8 may be classified as “high”, a value greater than or equal to 0.4 and less than 0.6 may be classified as “medium”, and arithmetic differences less than 0.4 may be classified as “low.”

In other implementations, the absolute value of the arithmetic difference may be used to determine the transition of care decision intervention priority score. In other implementations, other formulas and ranges can be used to define the different classifications without departing from the scope of the disclosure. In general, a greater difference in model outputs corresponds to a higher priority for a transition of care decision intervention, as the greater difference indicates that the likely decision to be made by the clinician based on historical data for the health care facility is likely to be different than what would be determined according to an independent expert. This is not to suggest that the independent expert would necessarily make a better decision for the particular patient, but only that greater the difference in model outputs, the greater the likelihood that the patient might benefit from additional thought being put into the final decision on the transition of care. If the difference between the model outputs are low, no special attention is needed, as the health care facilities' likely recommendation, according to the historical analysis is likely to be the same as the decision that would be made by an independent expert. Accordingly, a highest level of difference between model outputs can be considered to be a highest priority for a transition of care decision intervention, whereas a low difference in model outputs can be considered to indicate a low priority for a transition of care intervention. In some implementations, the ranges that define intervention priorities may not be based on raw difference scores, but instead on percentiles. For example, patients may be divided into four priority classifications based on a quartile in which the model output differences fall into. Differences falling in the top quartile are classified as highest priority, whereas patients falling into the lowest quartile are classified as low priority.

In some implementations, the transition of care decision intervention priority scores and/or the transition of care decision intervention priority score classification determined and outputted for one or more patient is associated with a respective transition of care decision intervention priority indicator. The transition of care decision intervention priority indicator may include symbols (such as a shape, a regular or flashing exclamation point, a colored icon, or a combination of any of these) that alert a healthcare practitioner of the patients' priority category. The priority indicators described herein are mere examples and any other suitable indicators, symbols, or alert systems that are capable of conveying the priority categories may be employed for this purpose. For example, the transition of care decision intervention priority indicator may be a grey square with a white dash indicating no priority, a purple square with an exclamation point indicating low priority, a green square with an exclamation point indicating medium priority, a yellow triangle with an exclamation point indicating high priority, or a red circle with a regular or flashing exclamation point indicating highest priority of transition of care decision intervention. The transition of care decision intervention priority indicators associated with the transition of care decision intervention priority score classification may be similarly outputted to a graphical user interface on the large-format computing device 105 and/or the small-format computing device 110.

The output data corresponding to the transition of care decision scores and/or the transition of care decision intervention priority indicators may be utilized by healthcare providers and/or benefits managers, insurers, and the like to determine the transition of care decision intervention plan for one or more patients.

FIGS. 2A-2E illustrate example block diagrams of systems for determining transition of care decision intervention priority scores for transition of care decision interventions using machine learning according to some implementations.

FIG. 2A is an example block diagram of a system 200a for determining transition of care decision intervention priority scores for transition of care decision interventions using machine learning according to some implementations. System 200a includes an input device 201 and an output device 202 coupled to a client 204. The client 204 includes a processor 206 and a memory 208 storing an application 210. The client 204 also includes a communications module 212 connected to network 214. System 200a also includes a server 216 which further includes a communications module 218, a processor 220 and a memory 222. The server 216 also includes one or more model training systems, such as a model training system 224. The model training system 224 includes some of the respective components used in performing similar training operations as the transition of care decision intervention system 125 of FIG. 1, except where indicated otherwise in the following description. In particular, the model training system 224 receives patient data as training input to generate and train at least two transition of care decision models. The server 216 also includes one or more execution systems, such as an execution system 226. The execution system 226 includes and utilizes the trained transition of care decision models of the model training system 224. The execution system 226 includes some of the respective components used in processing execution patient data and determining transition of care decision intervention priority scores using the transition of care decision models similar to the transition of care decision intervention system 125 shown in FIG. 1, except where indicated otherwise in the following description. Additional details of the different components included in model training system 224 and execution system 226 used herein to train the transition of care decision models and subsequently determine a transition of care decision intervention priority score, respectively, can be found below, e.g., in the description of FIGS. 2C and 2D below.

As shown in FIG. 2A, the system 200a includes an input device 201. The input device 201 receives user input and provides the user input to client 204. The input device 201 may include a keyboard, mouse, microphone, stylus, and/or any other device or mechanism used to input user data or commands to an application on a client, such as client 204. In some implementations, the input device 201 may include haptic, tactile or voice recognition interfaces to receive the user input, such as on a small-format device.

As shown in FIG. 2A, the system 200a also includes a client 204. The client 204 communicates via the network 214 with the server 216. The client 204 receives input from the input device 201. The client 204 can be, for example, a large-format computing device, such as large-format computing device 105 as shown in FIG. 1; a small-format computing device (e.g., a smartphone or tablet), such as small-format computing device 110 also shown in FIG. 1; a medical data device (e.g., a small or large-format device used in a healthcare setting to collect, manage or generate patient clinical data, demographic data, financial data, administrative data, health care utilization history, and any other patient record data as described in relation to the patient data 120 of FIG. 1 above), or any other similar device having appropriate processor, memory, and communications capabilities. The client 204 may be configured to receive, transmit, and store data associated with determining transition of care decision intervention priority score for one or more patients.

As further shown in FIG. 2A, the client 204 includes a processor 206 and a memory 208. The processor 206 operates to execute computer-readable instructions and/or process data stored in memory 208 and transmit instructions and/or data via the communications module 212. The memory 208 may store computer-readable instructions and/or data associated with obtaining and displaying transition of care decision intervention priority scores for one or more patients. For example, the memory 208 may include a database of patient data, such as patient records database 115 shown in FIG. 1. The memory 208 includes an application 210. The application 210 may be, for example, an application to receive user input or patient data for use in obtaining and displaying a transition of care decision intervention priority score for a given patient. In some implementations, the application 210 may receive user input or patient data for use in obtaining and displaying transition of care decision intervention priority scores for one or more patients in a given patient population. The application 210 may include textual and graphical user interfaces to receive patient data as input and to display output, including a transition of care decision intervention priority score and/or data corresponding to the transition of care decision scores for one or more patients. The data corresponding to the transition of care decision scores outputted on the application 210 may include any of the outputs described in relation to the large-format computing device 105 and/or the small-format computing device 110 of FIG. 1 above. In other implementations, the application 210 may further display as output, a transition of care decision intervention priority classification, including, for example, a no priority, a low priority, a medium priority, a high priority, and a highest priority category classification, as well as the corresponding transition of care decision intervention priority indicators, as also described in relation to FIG. 1 above.

The application 210 may include a number of configurable settings associated with triggering alerts or user notifications when a particular patient's transition of care decision intervention priority score or data corresponding to a particular patient's transition of care decision scores exceeds a threshold priority designation, e.g., high or highest priority designation. The application 210 may also display as output, transition of care decision intervention priority indicators associated with the intervention priority score and/or an intervention priority score classification determined and outputted for one or more patients as described in relation to FIG. 1 above. The transition of care decision intervention priority indicator may include symbols (such as a shape, a regular or flashing exclamation point, a colored icon, or a combination of any of these) that alert a healthcare practitioner of the patients' priority category. The priority indicators described herein are mere examples and any other suitable indicators, symbols, or alert systems that are capable of conveying the priority categories may be employed for this purpose. For example, the transition of care decision intervention priority indicator may be a grey square with a white dash indicating no priority, a purple square with an exclamation point indicating low priority, a green square with an exclamation point indicating medium priority, a yellow triangle with an exclamation point indicating high priority, or a red circle with a regular or flashing exclamation point indicating highest priority. Additionally, or alternatively, the application 210 may output, in a graphical user interface, a rank order of each patient in a given patient population based on the relative transition of care decision intervention priority score for each patient in the patient population. In some implementations, the application 210 may further output, in a graphical user interface, explanatory information underlying the determined transition of care decision intervention priority score for one or more patients. In some implementations, the explanatory information may include reasons and recommendations for an optimal service of care, care provider, and/or site of care for a particular patient based on the information corresponding to the patient's transition of care decision intervention. In some implementations, the explanatory information outputted by the application 210 may include clinical justifications for a particular patient's comorbidities, type, timing, nature, and degree of transition of care decision intervention and for prioritization of one patient over another in a patient population consistent with the urgency of the transition of care decision interventions at a given time in a given health care facility or health care system. In some implementations, such clinical justifications may include automated clinical justifications. Additionally, or alternatively, the explanatory information may include a list of transition of care discharge decision insights for a particular patient, such as discharge planning insights and/or health care utilization and recovery history. The discharge planning insights for a patient may be based on at least about past 3 months, 6 months, 9 months, 12 months or more of the available medical data for that patient. In some implementations, the discharge planning insights for a patient may be based on all of the available medical data for that patient. In some implementations, the discharge planning insights may comprise information, for example, information on the patient's diagnoses, procedures, and comorbidities, indicators of socio-behavioral need, markers of frailty and decreased mobility, episodes of hospitalization, emergency department visits, outpatient visits, and previous post-acute care service and provider utilization history, for example, at Home Health Agencies (HHA), Skilled Nursing Facilities (SNF), Inpatient Rehabilitation Facilities (IRF), Long-Term Acute Care hospitals (LTACHs), etc. In some implementations, discharge planning insights may also include key markers of outcomes including hospital readmission rates and readmission risk.

Additionally, or alternatively, the application 210 may also output, in a graphical user interface, recommendations consistent with the determined transition of care decision intervention priority score for one or more patients. In some implementations, the recommendation may include a personalized list for a particular patient including, but not limited to, a shortlist of recommended services of health care, care provider(s), facilities and the like, and/or agencies cross-checked with the patient's medical insurance, recommendations for follow-up and assessments, recommendations for clinical interventions by future providers, and recommended duration for a clinical intervention, institutionalization, series of home health care provider visits, and/or hospitalization. In some implementations, the application 210 may also output as recommendation, in a graphical user interface, at least one transition of care and/or discharge recommendation regarding an optimal service of care, care provider, and/or site of care, including but not limited to, recommendation for transition of care and/or discharge from emergency department (ED) to inpatient hospitalization, discharge from inpatient hospitalization to post-acute care services, providers, and sites including, for example, Home Health Agencies (HHA), Skilled Nursing Facilities (SNF), Inpatient Rehabilitation Facilities (IRF), Long-Term Acute Care hospitals (LTACHs), discharge from inpatient hospitalization to hospice care, discharge from post-acute care to outpatient services, discharge from post-acute care to home or home care, and discharge from intensive care unit (ICU) to inpatient hospitalization. In some implementations, the application 210 may also output other transition of care decision recommendations, e.g., for certain patient segments (including Medicaid) and any other clinical decision spaces more broadly involving transition of care.

As shown in FIG. 2A, the client 204 includes a communications module 212. The communications module 212 transmits the computer-readable instructions and/or patient data stored on or received by the client 204 via network 214. The network 214 connects the client 204 to the server 216. The network 214 can include, for example, any one or more of a personal area network (PAN), a local area network (LAN), a campus area network (CAN), a metropolitan area network (MAN), a wide area network (WAN), a broadband network (BBN), the Internet, and the like. Further, the network 214 may include, but is not limited to, any one or more of the following network topologies, including a bus network, a star network, a ring network, a mesh network, a star-bus network, tree or hierarchical network, and the like.

As further shown in FIG. 2A, the server 216 operates to receive, store and process the patient data communicated by client 204. In some implementations, the server 216 may receive patient data directly from one or more patient monitoring devices. The server 216 can be any device having an appropriate processor, memory, and communications capability for hosting a machine learning process. In certain aspects, one or more of the servers 216 may be located on-premises with client 204, or the server 216 may be located remotely from client 204, for example in a cloud computing facility or remote data center.

The server 216 includes a communications module 218 to receive the computer-readable instructions and/or patient data transmitted via network 214. The server 216 also includes one or more processors 220 configured to execute instructions that when executed cause the processors to determine a transition of care decision intervention priority score for one or more patients. The server 216 further includes a memory 222 configured to store the computer-readable instructions and/or patient data associated with determining a transition of care decision intervention priority score for one or more patients. For example, the memory 222 may store one or more computer models, such as the transition of care decision models generated during a machine learning process conducted by the transition of care decision intervention system 125 and the model training system 224. In some implementations, the memory 222 may store one or more machine learning algorithms that will be used to generate one or more transition of care decision models. In some implementations, the memory 222 may store patient data that is received from client 204 and is used as a training dataset (training input) in the machine learning process in order to train a transition of care decision model.

As shown in FIG. 2A, the server 216 includes one or more model training systems 224. A model training system 224 executes a machine learning process in which it receives patient data as training input and processes the patient data to train, using machine learning algorithms, at least two or more transition of care decision models, which can be subsequently used to determine transition of care decision intervention priority scores based on received patient data (shown in FIG. 1 as execution patient data). Additional details of the different components and functionality of each component included in the model training system 224 used herein to generate models to determine transition of care decision intervention priority scores can be found below, e.g., in the description of FIG. 2C below.

As further shown in FIG. 2A, server 216 includes one or more execution systems 226. The execution system 226 includes at least two or more trained transition of care decision models that were generated as a result of performing a machine learning process, for example the machine learning processes of the transition of care decision intervention system 125 (shown in FIG. 1) and of the model training system 224. The execution system 226 may receive patient data and process the patient data to output to the processor 220, a transition of care decision intervention priority score for one or more patients. Additional details of the different components and functionality of each component included in the execution system 226 used herein to determine a transition of care decision intervention priority score can be found below, e.g., in the description of FIG. 2D below.

In some implementations, the trained transition of care decision models produced in a machine learning process, may be subsequently included in an artificial intelligence system or application configured to receive patient data (execution patient data) and process the data to output a transition of care decision intervention priority score for one or more patients. In some implementations, the server 216 may create and store additional recommendations consistent with the determined transition of care decision intervention priority scores. In some implementations, the processor 220 may store the transition of care decision intervention priority score from the execution system 226 in memory 222. In some implementations, the memory 222 may store instructions to adjust or transform the received patient data based on the parameter input requirements of trained transition of care decision models. In other implementations, the outputted transition of care decision intervention priority scores may be forwarded to communications module 218 for transmission to the client 204 via network 214. Once received by the client 204, the outputted transition of care decision intervention priority scores may be transmitted to output device 202, such as a monitor, printer, portable hard drive or other storage device. In some implementations, the output device 202 may include specialized clinical diagnostic or laboratory equipment that is configured to interface with client 204 and may display the transition of care decision intervention priority scores.

FIG. 2B is an example block diagram of a system 200b for determining transition of care decision intervention priority scores for transition of care decision interventions using machine learning according to some implementations. System 200b includes a machine learning process configured on a model training server 228, and further includes a separate execution server 232 for utilizing the trained models, e.g., the trained models generated by the model training server 228. The individual components and functionality of each component of system 200b including an input device 201, an output device 202, client 204 including a processor 206, a memory 208 storing an application 210, and a communications module 212 connected to network 214, and a model training server 228 further including a communications module 218, a processor 220 and a memory 222 in FIG. 2B are identical to the corresponding components and functionality shown and described in relation to system 200a of FIG. 2A, with the exception that the model training server 228 shown in FIG. 2B only includes one or more model training systems 230 and does not include one or more execution systems 226 as shown in relation to server 216 of FIG. 2A. Instead, as shown in FIG. 2B, the system 200b includes an execution server 232 that is separate from the model training server 228. The execution server 232 also includes components and functionality similar to the server 216 shown in FIG. 2A, with the exception that the execution server 232 shown in FIG. 2B does not include a model training system, such as the model training system 224 shown in FIG. 2A.

FIG. 2C illustrates an example block diagram of a system 200c for machine learning models for determining transition of care decision intervention priority scores using a machine learning process configured on a model training server 236. The individual components and functionality of each component of system 200c including an input device 201, an output device 202, client 204 including a processor 206, a memory 208 storing an application 210, and a communications module 212 connected to network 214, and a model training server 236 including a communications module 218, a processor 220, a memory 222, and one or more model training systems 238 in FIG. 2C are identical to the corresponding components and functionality shown and described in relation to systems 200a of FIG. 2A, except where indicated otherwise in the following description. In particular, the model training server 236 as shown in FIG. 2C only includes one or more model training systems 238 and does not include an execution system 226 as shown in relation to server 216 of FIG. 2A.

As shown in FIG. 2C, system 200c includes a model training server 236. The model training server 236 includes similar components and operates similar to server 216 to receive, store and process the patient data communicated by client 204. The model training server 236 includes a communications module 218, a processor 220, a memory 222 and one or more model training systems 238, which include an optional feature selector 240, a historical decision-based model trainer 242 and an expert recommendation-based model trainer 246. In certain aspects, one or more model training server 236 can be located on-premises with client 204, or the model training server 236 may be located remotely from client 204, for example in a cloud computing facility or remote data center. In some implementations, the model training server 236 may be located in the same location as an execution server, for example, as shown and described in relation to the location of the model training server 228 and the execution server 232 of FIG. 2B. In other implementations, the model training server 236 may be located in a remote location, for example in a second data center that is separately located from the data center or hospital premises where an execution server is located.

As shown in FIG. 2C, the model training server 236 includes one or more model training systems 238, which implements a machine learning process. The model training system 238 includes an optional feature selector 240 (as shown in dashed line). The model training system 238 also includes a historical decision-based model trainer 242 and an expert recommendation-based model trainer 246, each of which generates respective models, i.e., historical decision-derived transition of care decision models 244 and expert recommendation-derived transition of care decision models 248 according to respective machine learning processes described below. The model training system 238 performs similar machine learning operations as the other machine learning systems disclosed herein (e.g., the transition of care decision intervention system 125 shown in FIG. 1 and the model training systems 224 and 230 shown in FIGS. 2A and 2B, respectively). Accordingly, the model training system 238, including its individual components, as shown and described in relation to the model training server 236 of FIG. 2C, may be used interchangeably with other model training systems disclosed herein (e.g., the model training systems 224 and 230 shown in FIGS. 2A and 2B, respectively) that performs similar machine learning operations in the machine learning systems and servers disclosed herein (e.g., the transition of care decision intervention system 125, server 216, and model training server 228).

In broad overview, the model training system 238 functions in the training aspect of a machine learning process. It receives patient data as training input and uses machine learning algorithms to generate and train at least two or more transition of care decision models, which can be subsequently used to determine at least two or more transition of care decision scores. The transition of care decision scores can be further processed to determine transition of care decision intervention priority scores for one or more patients.

As shown in FIG. 2C, the model training system 238, may additionally and optionally, include a feature selector 240 (as shown in dashed lines). When the feature selector 240 is optionally included in the model training system 238, the feature selector 240 operates in the machine learning process to receive patient data and select a subset of features from the patient data, which are provided as training input to a machine learning algorithm. In some implementations, the feature selector 240 receives patient data prior to or during the training portion of the machine learning process and may select subsets of inputs, also known as features for use in training the models and as inputs for the generated models. These subsets of inputs or features may include, but are not limited to, specific patient demographic characteristics, patient-individualized patterns of health care utilization including for skilled and unskilled care, facility-based care, and non-facility-based care, specific patient clinical characteristics including chief complaint, diagnoses, procedures, and comorbidities, indicators of socio-behavioral need, and markers of frailty and decreased mobility. The feature selector 240 can also combine and transform the selected subsets of inputs or features into supersets of features. Specifically, supersets of features may include, but are not limited to, derived indices that provide a quantitative, holistic measure of an individual patient's functional, clinical, and social status. A feature selection method, such as minimum-redundancy-maximum-relevance (e.g., Markov Blanket), lasso regression, ridge regression, forward selection, backward elimination, recursive feature elimination, random forest, etc., is then utilized to identify and provide specific sets or supersets of features as inputs to at least two or more different model trainers, e.g., a historical decision-based model trainer 242 and an expert recommendation-based model trainer 246, for generating respective models without overfitting such models.

In other implementations, the feature selector 240 may select a subset of features from the patient data that definitively correspond to a recommended transition of care decision intervention based on expert recommendations or best practice evidence, such that the machine learning algorithm will be trained to determine one or more transition of care decision scores based on the selected subset of features. In other implementations, the feature selector 240 may select a subset of features from the patient data that do not correspond to a recommended transition of care decision intervention based on expert recommendations or best practice evidence, but purely based on statistical correlations between data and decisions. By using a variety of training inputs, the machine learning process will generate trained models that are able to determine a patient's transition of care decision scores, which are subsequently used to determine a patient's transition of care decision intervention priority score, from a wide variety of disparate patient data.

As shown in FIG. 2C, the model training system 238 includes at least two or more different model trainers, e.g., a historical decision-based model trainer 242 and an expert recommendation-based model trainer 246, which receive patient data as training input to machine learning algorithms to generate the respective models, e.g., one or more historical decision-derived transition of care decision models 244 and one or more expert recommendation-derived transition of care decision models 248. In some implementations, when an optional feature selector 240 is included in the model training system 238, the feature selector 240 may provide selected features or supersets of features to the model trainers as inputs to a machine learning algorithm to generate the respective models. A wide variety of supervised machine learning classification and regression algorithms may be selected for use, such as algorithms, including but not limited to, support vector machine (SVM) classification and regression, artificial neural network (ANN) classification and regression, stochastic gradient descent classification and regression, ridge classification and regression, kernel ridge classification and regression, nearest neighbors classification and regression, decision tree classification and regression, random forest classification and regression, extra trees classification and regression, adaptive boosting classification and regression, ordinary least squares regression (OLSR), lasso regression, multi-task elastic net regression, logistic regression, multivariate adaptive regression splines (MARS), locally estimated scatterplot smoothing (LOESS), ordinal regression, Poisson regression, Gaussian process regression, and other machine learning methods employing Bayesian statistics, case-based reasoning, inductive logic programming, learning automata, learning vector quantization, informal fuzzy networks, conditional random fields, genetic algorithms (GA), or Information Theory.

In some implementations, the model training system 238 is configured with machine learning processes to train and output multiple historical decision-derived transition of care decision models 244 and multiple expert recommendation-derived transition of care decision models 248, which may have been trained in the machine learning process based on non-overlapping or partially overlapping sets of features.

In broad overview, the historical decision-derived transition of care decision model 242 is trained during the machine learning process using training input, as shown in patient data 120 in FIG. 1, which includes patient data from a relevant healthcare facility, system, or setting, and historical transition of care decisions made at that healthcare facility, system, or setting based on the respective patient data for the patients. On the other hand, the expert recommendation-derived transition of care decision model 246 is trained during the machine learning process using training input that includes the same or different patient data from the same or a different healthcare facility, healthcare system, or healthcare setting, and independent expert transition of care decision recommendations based on expert reviews of the relevant patient data for such patients. The trained transition of care decision models are then subsequently utilized for processing a wide variety of received execution patient data as input and determining the respective historical decision model-derived transition of care decision score and expert recommendation model-derived transition of care decision score for one or more patients.

During the training aspect of the machine learning process, the historical decision-based model trainer 242 and the expert recommendation-based model trainer 246 each receive patient data, including historical transition of care decisions and independent expert transition of care decision recommendations, respectively, as training input, or optionally, selected supersets of features as training input from the feature selector 240, and iteratively processes the training input using previously selected machine learning algorithms to assess performance of the resulting models. As the machine learning algorithm processes the training input, the model trainers learn patterns in the training input that map the machine learning algorithm variables to target output data (e.g., the transition of care decision intervention scores) and generates models, e.g., one or more historical decision-derived transition of care decision models 244 and one or more expert recommendation-derived transition of care decision models 248, that capture these relationships. Each model trainer may use a different feature set and employ a different machine learning algorithm to generate the respective models. For example, as shown in FIG. 2C, the historical decision-based model trainer 242 receives training input, including patient data and historical transition of care decisions based on that patient data, and employs a historical practice decisions-based machine learning algorithm to generate and train one or more historical decision-derived transition of care decision models 244. Similarly, the expert recommendation-based model trainer 246 receives training input, including patient data and independent expert transition of care decision recommendations based on that patient data, and employs an expert recommendations and best practice evidence-based machine learning algorithm to generate and train one or more expert recommendation-derived transition of care decision models 248. Thus, based on the different machine learning algorithms and processes utilized, the model trainers 242 and 246 of the model training system 238 may train and output respective models, e.g., one or more historical decision-derived transition of care decision models 244 and one or more expert recommendation-derived transition of care decision models 248, that perform in subsequently determining a patient's transition of care decision intervention priority score.

FIG. 2D illustrates an example block diagram of a system 200d for determining transition of care decision intervention priority scores using models that are generated by multiple or different machine learning processes. The individual components and functionality of each component of system 200d including an input device 201, an output device 202, client 204 including a processor 206, a memory 208 storing an application 210, and a communications module 212 connected to network 214, and an execution server 250 including a communications module 218, a processor 220, a memory 222, and one or more execution systems 252, shown in FIG. 2D are identical to the corresponding components and functionality shown and described in relation to system 200a of FIG. 2A, except where indicated otherwise in the following description. In particular, the execution server 250 as shown in FIG. 2D only includes one or more execution systems 252 and does not include a model training system 224 as shown in server 216 in FIG. 2A.

As shown in FIG. 2D, system 200d includes an execution server 250. The execution server 250 includes similar components and operates similar to server 216 to receive, store, and process the patient data communicated by client 204 as disclosed in relation to FIG. 2A. The execution server 250 includes a communications module 218, a processor 220, a memory 222 and one or more execution systems 252, which in turn includes trained transition of care decision models that output respective transition of care decision scores. The execution system 252 also includes a decision intervention priority score evaluation processor 262 that determines a transition of care decision intervention priority score 264. The execution system 252, including its individual components, may be used interchangeably with other execution systems disclosed herein (e.g., the execution systems 226 and 234 as shown in FIGS. 2A and 2B, respectively) that perform similar operations in determining transition of care decision intervention priority scores in the machine learning systems and servers disclosed herein.

In certain aspects, one or more of the execution server 250 can be located on-premises with client 204, or alternatively, the execution server 250 may be located remotely from client 204, for example in a cloud computing facility or remote data center. In some implementations, the execution server 250 may be located in the same location as model training server, for example, as shown and described in relation to the location of the model training server 228 and the execution server 232 of FIG. 2B. In other implementations, the execution server 250 may be located in a remote location, for example in a second data center that is separately located from the data center or hospital premises where the model training server is located.

As shown in FIG. 2D, the execution system 252 includes at least two or more trained transition of care decision models e.g., one or more historical decision-derived transition of care decision models 254 and one or more expert recommendation-derived transition of care decision models 258, that were generated as a result of performing a machine learning process. Upon receiving execution patient data from a client, for example the client 204, the trained transition of care decision models 254 and 258 process and output respective transition of care decision scores, e.g., a historical decision model-derived transition of care decision score 256 and an expert recommendation model-derived transition of care score 260, for one or more patients.

As further shown in FIG. 2D, the execution system 252 depicts a historical decision model-derived transition of care decision score 256 and an expert recommendation model-derived transition of care decision score 260 for one or more patients. The historical decision model-derived transition of care decision score 256 is outputted as a result of processing the execution patient data, for example from patient data 120 (shown in FIG. 1), through one or more trained historical decision-derived transition of care decision models 254. The expert recommendation model-derived transition of care decision score 260 is outputted as a result of processing the execution patient data, for example from patient data 120 (shown in FIG. 1), through the trained expert recommendation-derived transition of care decision models 258. The respective historical decision model-derived transition of care decision scores and expert recommendation model-derived transition of care decision scores are then used to determine a transition of care decision intervention priority score.

As shown in FIG. 2D, the execution system 252 further includes a decision intervention priority score evaluation processor 262. In some implementations, the decision intervention priority score evaluation processor 262 processes and outputs a transition of care decision intervention priority score 264 based on a degree of difference between the historical decision model-derived transition of care decision score 256 and the expert recommendation model-derived transition of care decision score 260, for a given patient. In other implementations, the decision intervention priority score evaluation processor 262 processes and outputs a transition of care decision intervention priority score 264 for each of one or more patients in a given patient population based on the respective degrees of difference between the historical decision model-derived transition of care decision scores 256 and the expert recommendation model-derived transition of care decision scores 260 for each patient.

In some implementations, the execution system 252 includes at least two or more trained expert recommendation-derived transition of care decision models 258 (e.g., trained based on evaluation from different experts), each of which determines a separate expert recommendation model-derived transition of care decision score 260, for one or more patients. The decision intervention priority score evaluation processor 262 then determines a single or an aggregated value for the expert recommendation model-derived transition of care decision score 260 by performing a desired aggregation function on a set of values of the expert recommendation model-derived transition of care decision score 260 that are determined by multiple expert recommendation-derived transition of care decision models 258 for each patient. An aggregation function can be any desired mathematical or statistical function that performs a calculation on a set of values and returns a single or an aggregated value, and includes, but is not limited to, an average or an arithmetic mean, a median, a mode, a count, a minimum, a maximum, a sum, a range, a standard deviation, a weighted mean, and the like. The decision intervention priority score evaluation processor 262 then processes and outputs a transition of care decision intervention priority score 264 based on the degree of difference between the historical decision model-derived transition of care decision score 256 and the single or aggregated value for the expert recommendation model-derived transition of care decision score obtained by the employed aggregation function, for each patient.

As further shown in FIG. 2D, the execution system 252 generates a transition of care decision intervention priority score 264 for one or more patients, which is transmitted to a large-format computing device 105, a small-format computing device 110 and/or the patient records database 115. The received patient transition of care decision intervention priority score 264 may be output to a graphical user interface on the large-format computing device 105 and/or the small-format computing device 110 as described in the description of FIG. 1 above. Other information related to the priority score, such as priority indicator or classification may be displayed in addition or instead of the raw priority score. The output patient transition of care decision intervention priority score 264 and/or related information may be utilized by healthcare providers to determine a transition of care decision intervention plan for a given patient and to select or perform preventative therapeutic interventions, post-acute care-related interventions, treatments, or actions as appropriate for the patient's determined transition of care decision intervention priority score.

Additionally, or alternatively, in some implementations, data corresponding to the transition of care decision scores for one or more patients is transmitted to computing devices and/or patient records database as described in the description of FIG. 1 above.

FIG. 2E is an example block diagram of a system 200e for transition of care decision intervention using machine learning according to some implementations. System 200e includes multiple or different machine learning processes for several different health care facilities or health care systems (HCF/S), e.g., HCF/S 1, HCF/S 2, and HCF/S 3. System 200e includes multiple clients, e.g., client HCF/S 1, client HCF/S 2, and client HCF/S 3, and corresponding machine learning processes configured on HCF/S 1 model training server 266, HCF/S 2 model training server 270, and HCF/S 3 model training server 274, respectively. System 200e also includes HCF/S 1 execution server 268, HCF/S 2 execution server 272, and HCF/S 3 execution server 276, for utilizing the respective trained models generated by the HCF/S 1 model training server 266, HCF/S 2 model training server 270, and HCF/S 3 model training server 274, respectively. The individual components and functionality of the client HCF/S 1, client HCF/S 2, and client HCF/S 3 are identical to the corresponding components and functionality of the client 204. The individual components and functionality of the model training servers 266, 270, and 270 of system 200e are identical to the corresponding components and functionality shown and described in relation to the model training server 236 of system 200c as shown in FIG. 2C, with the exception that system 200e is configured to include multiple or different model training servers, each of which is trained with a training input, for example from patient data 120, associated with a different health care facility or health care system. Such health care facility or health care system may include, but is not limited to, an emergency department, outpatient hospital facilities, outpatient services, acute inpatient hospitals, post-acute care providers including, for example, Home Health Agencies (HHA), Skilled Nursing Facilities (SNF), Inpatient Rehabilitation Facilities (IRF), Long-Term Acute Care hospitals (LTACHs), and hospice care. The individual components and functionality of the execution servers 268, 272, and 276 of system 200e are identical to the corresponding components and functionality shown and described in relation to the execution server 250 of system 200d as shown in FIG. 2D, with the exception that system 200e is configured to include multiple execution servers 268, 272, and 276, each of which is capable of determining a transition of care decision intervention priority score for the health care facility or health care system associated with the corresponding model training servers 266, 270, and 274.

In some implementations of the system 200e, at least one or more of the execution servers 268, 272, and 276 may include two or more trained expert recommendation-derived transition of care decision models 258, each of which determines a separate expert recommendation model-derived transition of care decision score 260, for one or more patients. A decision intervention priority score evaluation processor 262 included in each of the execution servers 268, 272, and 276 then determines a single or aggregated value for the expert recommendation model-derived transition of care decision score by performing a desired aggregation function on a set of values of multiple expert recommendation model-derived transition of care decision score 260 for one or more patients. An aggregation function can be any desired mathematical or statistical function that performs a calculation on a set of values and returns a single or an or aggregated value, and includes, but is not limited to, an average or an arithmetic mean, a median, a mode, a count, a minimum, a maximum, a sum, a range, a standard deviation, a weighted mean, and the like. The decision intervention priority score evaluation processor 262 further processes and outputs a transition of care decision intervention priority score 264 based on a degree of difference between the historical decision model-derived transition of care decision score 256 and the single or aggregated value for the expert recommendation model-derived transition of care decision score obtained by the employed aggregation function, for one or more patients.

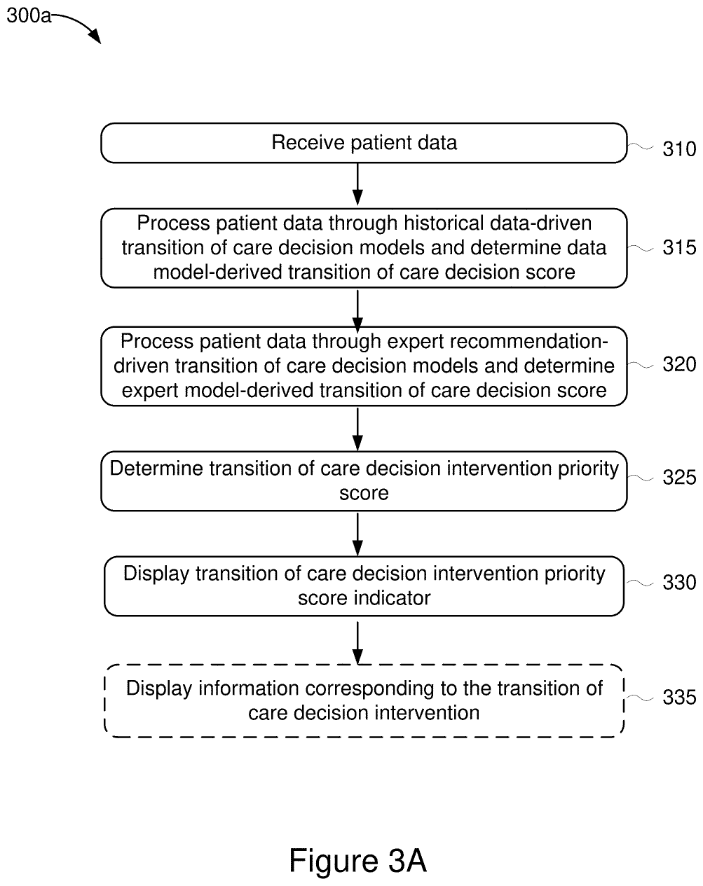

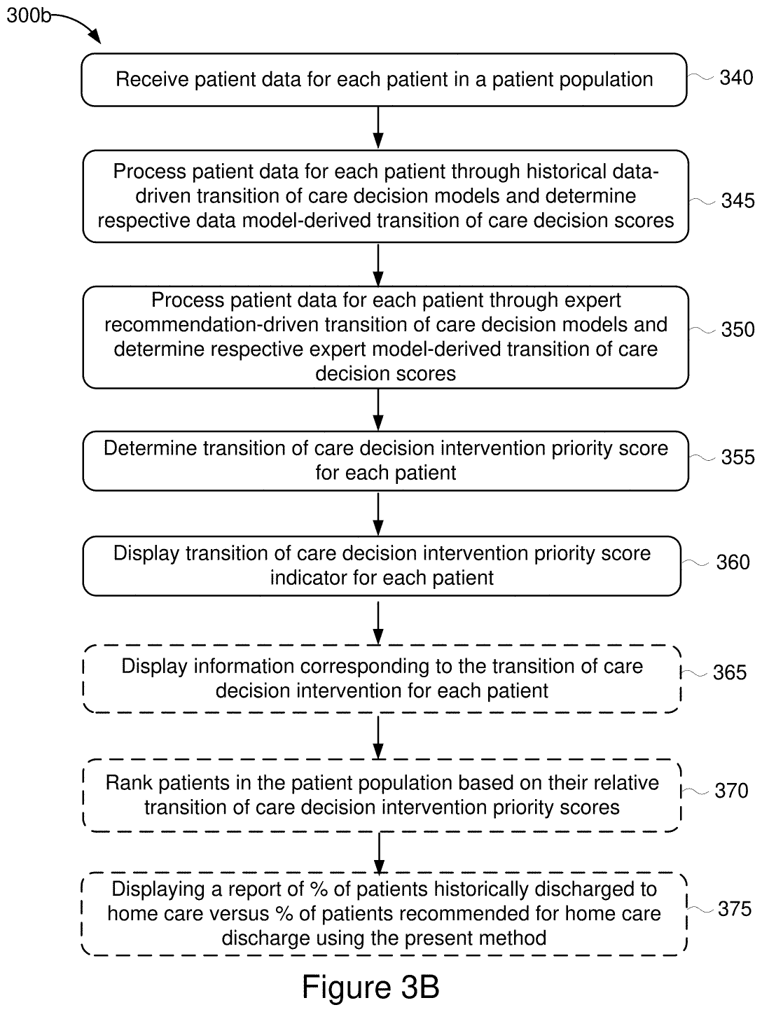

FIGS. 3A and 3B are flowcharts showing a method for determining transition of care decision intervention priority scores using historical decision-derived and expert recommendation-derived transition of care decision models derived from a machine learning process according to some implementations.

FIG. 3A illustrates an example method 300a for determining transition of care decision intervention priority score for one or more patients using historical decision-derived and expert recommendation-derived models derived from machine learning processes performed by servers 216, 228, 236, 250, 266, 270, and 274 of FIGS. 2A-2E. The method 300a includes receiving patient data (stage 310). The method further includes processing patient data through a historical decision-derived transition of care decision model and determining a historical decision model-derived transition of care decision score (stage 315). The method further includes processing patient data through an expert recommendation-derived transition of care decision models and determining an expert recommendation model-derived transition of care decision score (stage 320). The method also includes determining a transition of care decision intervention priority score (stage 325) and displaying a transition of care decision intervention priority score indicator (stage 330). The method may optionally include displaying a variety of information, such as explanatory information and recommendations, corresponding to the transition of care decision intervention (stage 335).

At stage 310, the method 300a begins by receiving patient data, such as execution patient data, at a server, such as server 216, shown in FIG. 2A. Patient data may be received from a variety of sources by the server. The server is configured with one or more trained transition of care decision models that have been previously trained in a machine learning process to determine transition of care decision scores for one or more patients. For example, patient data may be stored on one or more computing devices, such as the large-format computing device 105 and the small-format computing device 110 shown in FIG. 1. In addition, patient data may be stored in a network-accessible database, such as the patient records database 115 as shown in FIG. 1. In some implementations, the database may be on a client device, such as client device 204 shown in FIG. 2A.

The received patient data, which is similar to the execution patient data shown in FIG. 1, may include patient clinical data, demographic data, financial data, administrative data, health care utilization history, and other patient record data, collectively known as features, as described in details with relation to the patient data 120 (which also includes execution patient data) of FIG. 1 above. The received patient data may include one or more data elements or features that correspond to a specific clinical parameter or measurement obtained in a healthcare setting that may be useful in determining a particular type of patient's transition of care decision intervention priority score, for example, specific patient demographic characteristics, patient's historic health care utilization, recovery history, and current hospital presentation, comorbidities, socio-behavioral need, markers of frailty, and decreased mobility.

The patient data may include encounter data such as patient identifiers, the patient's date of birth, and the dates and times or admission or discharge from the hospital. The encounter data may include information on the specific nature of a patient's interaction, including any sporadic encounters, with the healthcare system, e.g., emergency department, inpatient, outpatient, and post-acute care. The encounter data may also include all relevant accompanying information for each episode of care. A discrete encounter begins when a patient is first presented to a specific setting of health care (e.g., ED, inpatient, outpatient, post-acute, home, hospice, etc.) and ends when they transition to a different setting of care. As such, encounters may be sporadic in nature. In some implementations, the processors disclosed herein, such as the processor 206 and 220, may identify a specific patient who participated in an encounter and combine together any current and previous encounters for an individual patient into a longitudinal patient history.

The patient data may also include chart data identifying time stamps and numerical values for any treatments or actions taken by healthcare providers. The patient data may include laboratory data identifying time stamps and numerical values for the results of any diagnostic tests performed on the patient. The patient data may further include medication data identifying medication type, medication dosage, and time stamps for when the medication was administered to the patient. The patient data may also include diagnosis and procedure codes that might provide information on clinical, functional, or social risk factors and time stamps for when these codes have been logged.

At stage 315, a server, such as the execution server 250, processes the execution patient data through a historical decision-derived transition of care decision model and determines the historical decision model-derived transition of care decision score. The received execution patient data is processed using one or more trained historical decision-derived transition of care decision models, such as the trained historical decision-derived transition of care decision models 254 shown in FIG. 2D, to determine the historical decision model-derived transition of care decision score, such as the historical decision model-derived transition of care decision score 256 shown in FIG. 2D, for one or more patients.

At stage 320, the execution server 250 processes the execution patient data through an expert recommendation-derived transition of care decision model and determines the expert-model derived transition of care decision score. The received execution patient data is processed using one or more trained expert recommendation-derived transition of care decision models, such as the trained expert recommendation-derived transition of care decision models 258 shown in FIG. 2D, to determine the expert recommendation model-derived transition of care decision score, such as the expert recommendation model-derived transition of care decision score 260 shown in FIG. 2D, for one or more patients.

In some implementations, prior to determining the transition of care decision scores by running the execution patient data through the transition of care decision models, the execution patient data can be filtered to identify patients for which the transition of care decision can be definitively determined based on one or more definitive transition of care decision rules. Such rules may be defined by a variety of organizations, such as government agencies, insurance companies, or health systems or facilities. It is unnecessary to process patients' data that satisfy these rules as the rules dictate a definite answer between the possible transition of care options. Such rules can avoid having the data processing by one or both of the historical decision-derived transition of care decision model and the expert recommendation-derived transition of care decision model. Similar filters may be employed in the model training process to limit the number of cases an expert needs to review to develop the training set used to train the expert recommendation-derived transition of care model. In situations where execution data for a patent triggers one or more of these filters, the transition of care score for the applicable models can be set to 0.0 or 1.0, depending on the value dictated by the rule in light of the patient data. In some implementations, the triggering of the filter rule and the corresponding rule output can also be communicated back to the client 204 for outputting to a clinician or other decision maker.

At stage 325, the execution server 250 determines the transition of care decision intervention priority scores. The historical decision model-derived transition of care decision score and the expert recommendation model-derived transition of care score determined at stage 315 and 320, respectively, are processed at stage 325 by a decision intervention priority score evaluation processor, such as the decision intervention priority score evaluation processor 262 shown in FIG. 2D. The decision intervention priority score evaluation processor 262 processes the determined historical decision model-derived transition of care decision score 256 and expert recommendation model-derived transition of care score 258 and outputs a transition of care decision intervention priority score 264 based on the degree of difference between the historical decision model-derived transition of care decision score 256 and the expert recommendation model-derived transition of care decision score 260. In some implementations, the expert recommendation model-derived transition of care score may be determined by averaging the values for multiple expert recommendation model-derived transition of care decision scores 260 determined by multiple different expert recommendation-derived transition of care decision models 258 of stage 320. In some implementations, at stage 325, the data corresponding to the transition of care decision scores is also outputted. The data corresponding to the transition of care decision scores may further include any of the outputs, e.g., raw transition of care decision scores, raw scores corresponding to a probability value, and/or scores for each model normalized to a common scale, as described for FIG. 1. In some implementations, in addition to, or instead of the raw model outputs, the data corresponding to the transition of care decision scores may include a transition of care decision intervention priority score and any other outputs, e.g., an arithmetic or a percentage difference between the two transition of care decision scores and/or a classification of the level of difference (e.g., highest level of difference, high level of difference, medium level of difference, or low level of difference) as described for FIG. 1 above. In some implementations, the model output scores are normalized and a priority classification based on various ranges is further output as described for FIG. 1.

In some implementations, at stage 325, the transition of care decision intervention priority classification may include a no priority, a low priority, a medium priority, a high priority, and a highest priority category classification associated with each of the corresponding absolute ranges of differences or the percentage-based score assessments. For example, a patient's transition of care decision intervention priority may be classified as low and/or no priority for an absolute range of difference of 0-10, medium priority for an absolute range of difference of 11-20, a high priority for an absolute range of difference of 21-30, and a highest priority for an absolute range of difference of 31 and above, between the two transition of care decision scores. In another implementation, a patient's transition of care decision intervention priority may be classified based on an absolute range (between 0.0 and 1.0) of difference of values between the two transition of care decision scores, where a value greater than or equal to 0.8 may be classified as “highest priority”, a value greater than or equal to 0.6 and less than 0.8 may be classified as “high priority”, a value greater than or equal to 0.4 and less than 0.6 may be classified as “medium priority”, and a value less than 0.4 may be classified as “low and/or no priority.” In other implementations, a patient's transition of care decision intervention priority may be classified as low and/or no priority for a percentage range change of 1-10%, medium priority for 11-20% change, a high priority for 21-30% change, and a highest priority for 31% and above, between the two transition of care decision scores.

At stage 330, the execution server 250 displays a transition of care decision intervention priority score indicator. The transition of care decision intervention priority scores and/or the transition of care decision intervention priority score classification determined and outputted for one or more patient at stage 325 is associated with a respective transition of care decision intervention priority indicator. The transition of care decision intervention priority score indicator displayed at stage 330 may reflect the patient priority level associated with the data corresponding to the transition of care decision scores. The indicator may include symbols (such as a shape, a regular or flashing exclamation point, a colored icon, or a combination of any of these) that alert a healthcare practitioner of the patients' priority category. The indicators described herein are mere examples and any other suitable indicators, symbols, or alert systems that are capable of conveying the priority categories may be employed for this purpose. For example, the indicator may be a grey square with a white dash indicating no priority, a purple square with an exclamation point indicating low priority, a green square with an exclamation point indicating medium priority, a yellow triangle with an exclamation point indicating high priority, or a red circle with a regular or flashing exclamation point indicating highest priority. In some implementations, the determined transition of care decision intervention priority score, raw data corresponding to the transition of care decision scores, and/or the transition of care decision intervention priority classification may be output to a memory located on the server, for example memory 222 on server 250 as shown in FIG. 2D. In other implementations, the determined transition of care decision intervention priority score and/or the data corresponding to the transition of care decision scores, and/or the transition of care decision intervention priority classification may be stored in a database, such as patient records database 115 shown in FIG. 1. In this example, the patient records database 115 may be configured on client 204 and/or on servers 216, 228, 236, 266, 270, and 274.

In some implementations, the execution server 250 may output the determined transition of care decision intervention priority score and/or the data corresponding to the transition of care decision scores to client 204 shown in FIGS. 2A-2E. In some implementations, the client 204 may include a graphical user interface to display the transition of care decision intervention priority score indicators reflecting the patient priority level, e.g., no priority, low priority, medium priority, high priority, or highest priority, associated with the determined transition of care decision intervention priority score and/or the data corresponding to the transition of care decision scores. For example, application 210 on client 204 may include a graphical user interface to display the transition of care decision intervention priority score indicators, the determined transition of care decision intervention priority score, and/or the data corresponding to the transition of care decision scores. For example, the client 204 may be a monitor in the emergency department (ED), intensive care unit (ICU), or ward of a hospital facility used to display patient data. The transition of care decision intervention priority score indicators, the determined transition of care decision intervention priority score, and/or the data corresponding to the transition of care decision scores may be displayed in a graphical user interface on the monitor to enable healthcare practitioners in the ED, ICU or ward to view patient's transition of care decision intervention priority score and/or the data corresponding to the transition of care decision scores. Displaying the determined transition of care decision intervention priority score and/or the data corresponding to the transition of care decision scores allows healthcare practitioners to determine a transition of care decision intervention plan for a patient and to select or perform preventative therapeutic interventions, post-acute care-related interventions, treatments, or actions as appropriate for the patients' determined transition of care decision intervention priority score.

In other implementations, the server 216 outputs the determined transition of care decision intervention priority score and/or the data corresponding to the transition of care decision scores to the client 204, and the client 204 may further store the determined transition of care decision intervention priority score and/or the data corresponding to the transition of care decision scores in memory 208. In some implementations, the server 216 may output the determined transition of care decision intervention priority score and/or the data corresponding to the transition of care decision scores to the client 204 and the client 204 may further output the determined transition of care decision intervention priority score and/or the data corresponding to the transition of care decision scores to output device 202.

The method 300a may optionally include stage 335 for displaying a variety of information, such as explanatory information and recommendations, corresponding to the transition of care decision scores. In some implementations, such information may include explanatory information underlying the determined transition of care decision intervention priority score. In some implementations, the explanatory information may include reasons and recommendations for an optimal service of care, care provider, and/or site of care for a particular patient based on the patient's transition of care decision scores. In some implementations, the explanatory information displayed at stage 335 may include clinical justifications for a particular patient's comorbidities, type, timing, nature, and degree of transition of care decision intervention. In some implementations, such clinical justifications may include automated clinical justifications. Additionally, or alternatively, the explanatory information displayed at stage 335 may include a list of transition of care discharge decision insights for a particular patient, such as discharge planning insights and/or health care utilization and recovery history. In some implementations, the discharge planning insights for a patient may be based on at least about past 3 months, 6 months, 9 months, 12 months or more of the available medical data for that patient. In some implementations, the discharge planning insights for a patient may be based on all of the available medical data for that patient. In some implementations, the discharge planning insight may include additional information, for example, information on the patient's diagnoses, procedures, and comorbidities, indicators of socio-behavioral need, markers of frailty and decreased mobility, episodes of hospitalization, emergency department visits, outpatient visits, and previous post-acute care provider utilization history, for example, at Home Health Agencies (HHA), Skilled Nursing Facilities (SNF), Inpatient Rehabilitation Facilities (IRF), Long-Term Acute Care hospitals (LTACHs), etc. In some implementations, discharge planning insights may also include key markers of outcomes including hospital readmission rates and risk.

Additionally, or alternatively, the information displayed at stage 335 may also include recommendations consistent with the determined transition of care decision scores for one or more patients. In some implementations, the recommendation may include a personalized list for a particular patient that includes, but is not limited to, a shortlist of recommended services of health care, care provider(s), facilities and the like, and/or agencies cross-checked with the patient's medical insurance, recommendations for follow-ups and assessments, recommendations for clinical interventions by future providers, and recommended duration for a clinical intervention, institutionalization, series of home health care provider visits, and/or hospitalization. In some implementations, the recommendation may include at least one transition of care and/or discharge recommendation regarding a preferred future site of care, including but not limited to, recommendation for transition of care and/or discharge from emergency department to inpatient hospitalization, inpatient hospitalization to post-acute care facilities or services, discharge from inpatient hospitalization to hospice care, discharge from post-acute care to outpatient services, discharge from post-acute care to home or home care, and discharge from intensive care unit (ICU) to inpatient care. In some implementations, the recommendation may also include other transition of care decision recommendations, e.g., for certain patient segments (including Medicaid) and any other clinical decision spaces more broadly involving transition of care.

In some implementations, the explanatory information and recommendations described above may be displayed in a graphical user interface, output and stored in similar manners and implementations as described at stage 330 in relation to the transition of care decision intervention priority score indicators, the determined transition of care decision intervention priority score, and/or the data corresponding to the transition of care decision scores. Displaying the variety of explanatory information and recommendations at stage 335 allows healthcare practitioners to determine a transition of care decision intervention plan for a given patient and to select or perform preventative therapeutic interventions, post-acute care-related interventions, treatments, or actions as appropriate for the patients' determined transition of care decision intervention priority score.

FIG. 3B illustrates an example method 300b for determining transition of care decision intervention priority score for each patient in a given patient population using historical decision-derived and expert recommendation-derived transition of care decision models generated by the machine learning processes performed by servers 216, 228, 236, 250, 266, 270, and 274 of FIGS. 2A-2E. The method 300b includes receiving patient data for each patient in a given patient population for a health care facility or health care system (stage 340). The method further includes processing patient data for each patient in the given patient population through historical decision-derived transition of care decision models and determining respective historical decision model-derived transition of care decision score for each patient in the given patient population (stage 345). The method also includes processing patient data for each patient in the given patient population through expert recommendation-derived transition of care decision models and determining respective expert recommendation model-derived transition of care decision scores for each patient in the given patient population (stage 350). The method further includes determining respective transition of care decision intervention priority scores for each patient in the given patient population (stage 355) and displaying respective transition of care decision intervention priority score indicator for each patient in the given patient population (stage 360). The method may optionally include displaying a variety of information, such as explanatory information and recommendations, corresponding to the transition of care decision scores for each patient in the given patient population (stage 365). The method may optionally also include ranking the patients in the patient population based on their relative transition of care decision intervention priority score (stage 370) and displaying a report indicating the number of patients historically discharged to home care versus the number of patients recommended for home care discharge using the systems and methods disclosed herein (stage 375).

The stages and operations performed by each stage shown and described in relation to method 300b in FIG. 3B are identical to the corresponding stages and operations shown and described in relation to system 300a of FIG. 3A, except where indicated otherwise in the following description. In particular, stages 340-365 of method 300b are identical to the corresponding stages 310-335 of method 300a, except that stages 340-365 include iteratively receiving and processing patient data for each patient in a given patient population for a health care facility or health care system to determine respective transition of care decision intervention priority scores and display respective transition of care decision intervention priority score indicators and information corresponding to the transition of care decision intervention for each patient in the given patient population at a given time. In addition, method 300b may optionally include additional stages 370 and 375, which are not included in method 300a.

The method 300b may optionally include stage 370. At stage 370, a server, such as the execution server 250 shown in FIG. 2D, ranks the patients in the patient population based on their relative transition of care decision intervention priority scores derived at stage 355. Ranking the patients in the patient population based on their relative transition of care decision intervention priority scores allows healthcare practitioners to quickly scan a list of patients to determine the type, timing, nature, and degree of transition of care decision intervention recommended for each patient and to prioritize one patient over another in a given patient population consistent with the urgency of the transition of care decision interventions at a given time. In some implementations, such ranking of the patients in the patient population based on their relative transition of care decision intervention priority score may be displayed in a graphical user interface, output and stored in similar manners and implementations as described at stage 330 of method 300a (shown in FIG. 3A).

The method 300b may optionally also include stage 375. At stage 375, the execution server 250 displays a report indicating the percentage of patients historically discharged to home care from a particular health care facility or health care system (based on patient data, for example the patient data 120 as shown in FIG. 1), as compared to the percentage of patients currently recommended for home care discharge from the same health care facility or health care system using the systems and methods disclosed herein. Displaying such a report may allow healthcare practitioners to evaluate the impact of the transition of care decision intervention systems and models disclosed herein on the healthcare practitioners' decisions. In some implementations, the report of stage 375 may be displayed in a graphical user interface, output and stored in similar manners and implementations as described at stage 330 of method 300a (shown in FIG. 3A).

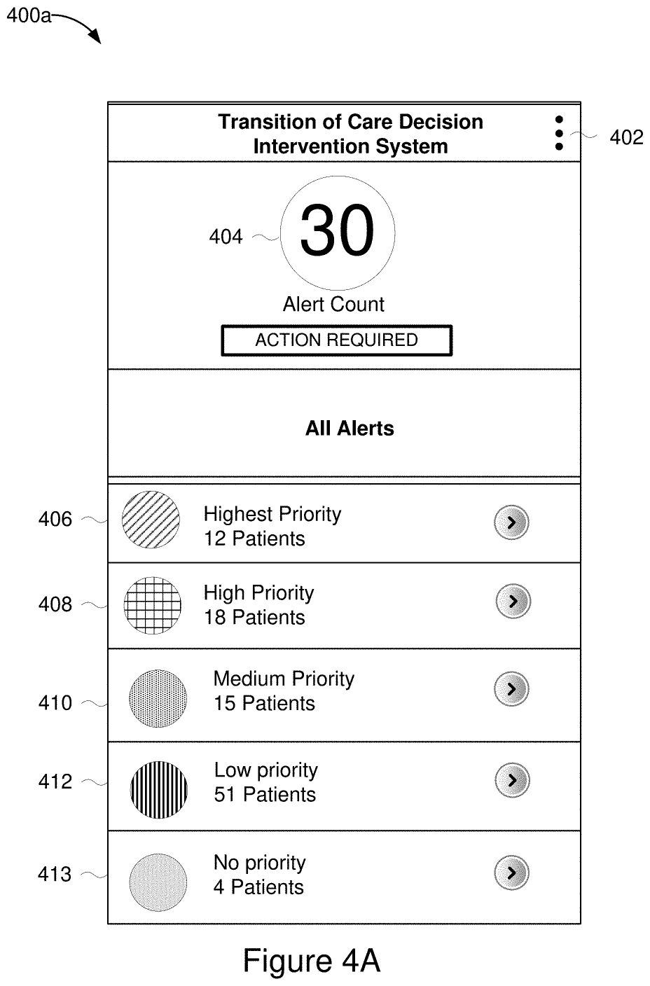

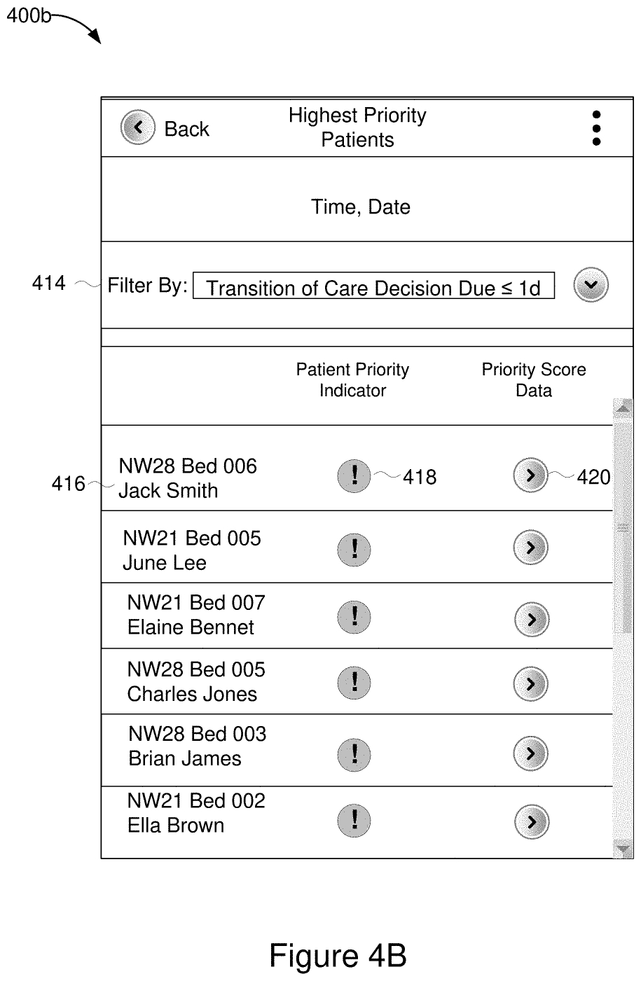

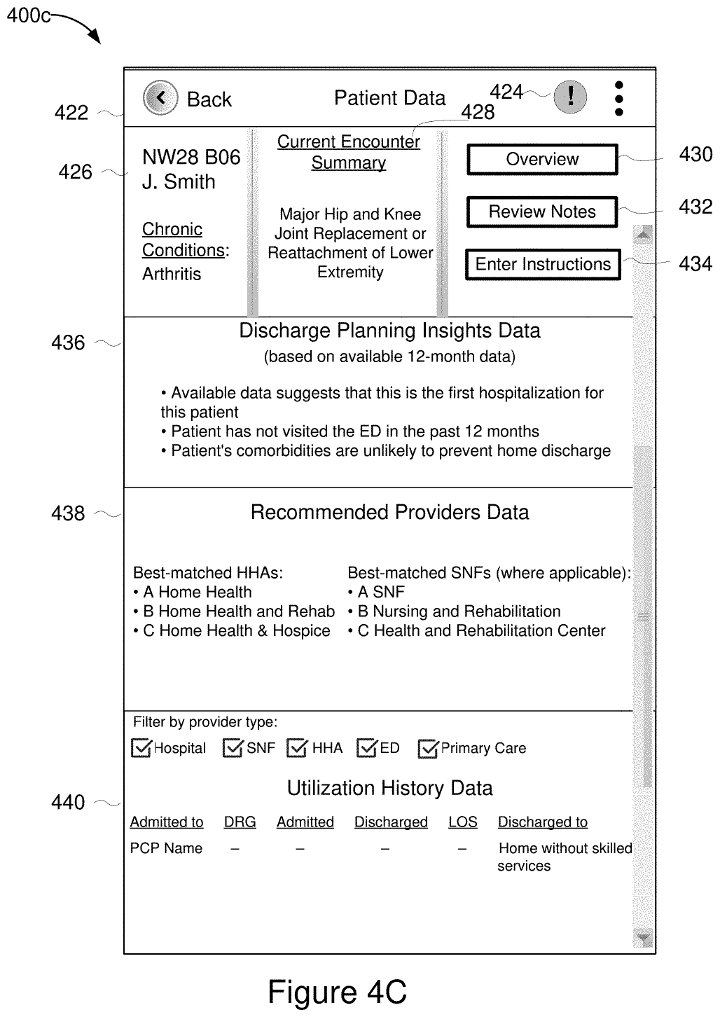

FIGS. 4A-4C illustrate example user interfaces for displaying and interacting with transition of care decision intervention priority scores according to some implementations. The user interfaces shown in FIGS. 4A-4C allow a healthcare practitioner to receive transition of care decision intervention priority scores, data corresponding to the transition of care decision scores, and/or information corresponding to transition of care decision intervention, for one or more patients and take actions based on such transition of care decision intervention priority scores, data, and/or information. In some implementations, the computing device is a small-format computing device displaying the user interfaces. The small-format computing device displaying the user interfaces may be a tablet, smart phone, or other similar small-format computing device used to maintain, input, receive, display, and/or transmit patient data. In other implementations, the computing device is a large-format computing device displaying the user interfaces. The large-format computing device displaying the user interfaces may be a large-format computer, a computing terminal with a display, or other similar non-small-format computing devices used to maintain, input, receive, display, and/or transmit patient data.

In some implementations the small-format and large-format computing device may be a clinical diagnostic device configured with a display, such as an electrocardiogram (EKG), a non-invasive ventilator, or a monitoring system in the emergency department (ED) or an intensive care unit (ICU). The clinical diagnostic device may be further configured to display the determined transition of care decision intervention priority scores and data corresponding to the transition of care decision scores for one or more patients on a user interface. In some implementations, the clinical diagnostic device may receive inputs of patient data and transmit patient data that are specifically related to a particular patient data feature used to determine transition of care decision intervention priority scores.

FIG. 4A illustrates an example user interface 400a for displaying and interacting with transition of care decision intervention based on patient's transition of care decision intervention priority score, related data, and/or information on a computing device. User interface 400a includes a system settings element 402, an alert count indicator 404, a highest priority patient count indicator 406, a high priority patient count indicator 408, a medium priority patient count indicator 410, a low priority patient count indicator 412, and a no priority patient count indicator 413.

As shown in FIG. 4A, the user interface 400a provides healthcare practitioners with a graphical display identifying the transition of care decision intervention priority scores, priority score data, and/or patient priority categories. The user interface 400a includes a system settings element 402, which is an interactive element for accessing system settings or configuration details. The user interface 400a also includes an alert count indicator 404. The alert count indicator 404 may inform the healthcare practitioner of the total number of highest and high priority patients at a given time based on the transition of care decision intervention priority scores. Additionally, or alternatively, the alert count indicator 404 may also be configured to identify the number of time-critical or prioritized transition of care decision interventions that need to be performed urgently in order to maximize the likelihood of beneficial impact for the patients requiring such time-critical transition of care decision intervention. As an example, the alert count indicator 404 shown in FIG. 4A indicates there are a total of 30 patients with highest and high priorities at a given time, and that healthcare practitioners should review the individual patient's data to determine the appropriate next course of action. By selecting or clicking on the alert count indicator 404, the user interface 400a may present to the healthcare practitioner a list of the 30 patients with highest and high priorities at the given time based on the patient's transition of care decision intervention priority score, related data, and/or information. In other implementations, the alert count indicator 404 may be configured to represent all patient priority categories that have been generated. In such implementations, by selecting or clicking on the alert count indicator 404, the user interface 400a may present to the healthcare practitioner a list of all patients rank ordered based on their relative transition of care decision intervention priority scores. Using this displayed data, a team of healthcare practitioners may better manage the transition of care decision intervention plan, options, and timing for a given patient based on the transition of care decision intervention priority score, related data, and/or information.

User interface 400a includes a highest priority patient count indicator 406. The highest priority patient count indicator 406 provides data to the healthcare practitioner about the number of patients whose determined transition of care decision intervention priority score, related data, and/or information indicate that such patients are the best candidates for a transition of care decision intervention. The assignment of a patient to the highest priority category may be based on a patient's transition of care decision intervention priority score exceeding a user configured threshold value and/or based on the likelihood of impact, feasibility, and time-criticality of the transition of care decision interventions analyzed based on the transition of care decision intervention priority score data. As used herein, the term “likelihood of impact” is defined as the extent to which a transition of care decision intervention is predicted to improve patient outcomes and/or reduce total costs of care. As used herein, the term “feasibility” is defined as the likelihood that a clinical care team can actually change a patient's plan of care by executing a transition of care decision intervention. As used herein, the term “time-criticality of the transition of care decision interventions” is defined as interventions that need to be performed urgently in order to maximize the likelihood of the beneficial impact for the patient requiring such time-critical transition of care decision intervention. The highest priority patient count indicator 406 may include any symbol (such as a shape, a regular or flashing exclamation point, a colored icon, or a combination of any of these) that alerts a healthcare practitioner of the patients' priority category. Any other suitable indicators, symbols, or alert systems that are capable of conveying the priority category may be employed for this purpose. In some implementations, the highest priority patient count indicator 406 may be accompanied by a colored icon (such as a red circle). In other implementations the highest priority patient count indicator 406 may be accompanied by an animated icon (such as a flashing exclamation point). The highest priority patient count indicator 406 may also include an interactive element, which when selected in the user interface will provide the healthcare practitioner with the list of patients determined to be in the highest priority category based on their transition of care decision intervention priority score or transition of care decision intervention priority score data. For example, as shown in user interface 400a, the highest priority patient count indicator 406 includes an icon displaying a right pointing chevron within a circle, which when selected displays the list of patients in the highest priority category in the user interface 400a.

User interface 400a includes a high priority patient count indicator 408. The high priority patient count indicator 408 provides data to the healthcare practitioner about the number of patients whose determined transition of care decision intervention priority score, related data, and/or information indicate that such patients are the second best candidates for a transition of care decision intervention. The assignment of a patient to the high priority category may be based on a patient's transition of care decision intervention priority score exceeding a user configured threshold value and/or based on the likelihood of impact, feasibility, and time-criticality of the transition of care decision interventions analyzed based on the transition of care decision intervention priority score data. The high priority patient count indicator 408 may include any symbol (such as a shape, a regular or flashing exclamation point, a colored icon, or a combination of any of these) that alerts a healthcare practitioner of the patients' priority category. Any other suitable indicators, symbols, or alert systems that are capable of conveying the priority category may be employed for this purpose. In some implementations, the high priority patient count indicator 408 may be accompanied by a colored icon (such as a yellow triangle). In other implementations the high priority patient count indicator 408 may be accompanied by an animated icon (such as an exclamation point). The high priority patient count indicator 408 may also include an interactive element, which when selected in the user interface will provide the healthcare practitioner with the list of patients determined to be in the high priority category based on their transition of care decision intervention priority score or transition of care decision intervention priority score data. For example, as shown in user interface 400a, the high priority patient count indicator 408 includes an icon displaying a right pointing chevron within a circle, which when selected displays the list of patients in the high priority category in the user interface 400a.

User interface 400a includes a medium priority patient count indicator 410. The medium priority patient count indicator 410 provides data to the healthcare practitioner about the number of patients whose determined transition of care decision intervention priority score, related data, and/or information indicate that such patients are the third best candidates for a transition of care decision intervention. The assignment of a patient to the medium priority category may be based on a patient's transition of care decision intervention priority score exceeding a user configured threshold value and/or based on the likelihood of impact, feasibility, and time-criticality of the transition of care decision interventions analyzed based on the transition of care decision intervention priority score data. The medium priority patient count indicator 410 may include any symbol (such as a shape, a regular or flashing exclamation point, a colored icon, or a combination of any of these) that alerts a healthcare practitioner of the patients' priority category. Any other suitable indicators, symbols, or alert systems that are capable of conveying the priority category may be employed for this purpose. In some implementations, the medium priority patient count indicator 410 may be accompanied by a colored icon (such as a green square). In other implementations the medium priority patient count indicator 410 may be accompanied by an animated icon (such as an exclamation point). The medium priority patient count indicator 410 may also include an interactive element, which when selected in the user interface will provide the healthcare practitioner with the list of patients determined to be in the medium priority category based on their transition of care decision intervention priority score or transition of care decision intervention priority score data. For example, as shown in user interface 400a, the medium priority patient count indicator 410 includes an icon displaying a right pointing chevron within a circle, which when selected displays the list of patients in the medium priority category in the user interface 400a.

User interface 400a includes a low priority patient count indicator 412. The low priority patient count indicator 412 provides data to the healthcare practitioner about the number of patients whose determined transition of care decision intervention priority score, related data, and/or information indicate that such patients are not good candidates for a transition of care decision intervention and are therefore assigned a low priority for a transition of care decision intervention. In some implementations, the assignment of a patient to the low priority category may be based on a patient's transition of care decision intervention priority score being below a user configured threshold value and/or based on the lack of likelihood of impact, feasibility, and time-criticality of the transition of care decision interventions analyzed based on the transition of care decision intervention priority score data. The low priority patient count indicator 412 may include any symbol (such as a shape, a regular or flashing exclamation point, a colored icon, or a combination of any of these) that alerts a healthcare practitioner of the patients' priority category. Any other suitable indicators, symbols, or alert systems that are capable of conveying the priority category may be employed for this purpose. In some implementations, the low priority patient count indicator 412 may be accompanied by a colored icon (such a purple square). In other implementations, the low priority patient count indicator 412 may be accompanied by an exclamation point. As described above in relation to the high risk patient count indicator 406, the low priority patient count indicator 412 includes an icon displaying a right pointing chevron within a circle, which when selected in the user interface will provide the healthcare practitioner with the list of patients determined to be in the low priority category.

User interface 400a includes a no priority patient count indicator 413. The no priority patient count indicator 413 provides data to the healthcare practitioner about the number of patients whose determined transition of care decision intervention priority score, related data, and/or information indicate that such patients are not candidates for a transition of care decision intervention and are therefore not prioritized for a transition of care decision intervention. The no priority patient count indicator 413 provides data to the healthcare practitioner about the number of patients for whom there is insufficient patient data available to determine transition of care decision intervention priority scores. For example, patients who are newly admitted to the ED or ICU may not have enough associated patient data (also known as execution patient data) to be used for determining their transition of care decision intervention priority scores at a given time. As more data is generated for the patient, the transition of care decision intervention priority scores may be determined for the patient and the patient may be assigned to the low, medium, high, or highest priority categories based on the determined transition of care decision intervention priority scores at a given time. The no priority patient count indicator 413 may include any symbol (such as a shape, a regular or flashing exclamation point, a colored icon, or a combination of any of these) that alerts a healthcare practitioner of the patients' priority category. Any other suitable indicators, symbols, or alert systems that are capable of conveying the priority category may be employed for this purpose. In some implementations, the no priority patient count indicator 413 may be accompanied by a colored icon (such a grey square). In other implementations, the no priority patient count indicator 413 may be accompanied by a dash. As described above in relation to the high risk patient count indicator 406, the no priority patient count indicator 413 includes an icon displaying a right pointing chevron within a circle, which when selected in the user interface will provide the healthcare practitioner with the list of patients determined to be in the no priority category.

FIG. 4B illustrates an example user interface 400b on a computing device for displaying and interacting with patients who have been assigned to a particular priority category, for example the highest priority category, based on the patient's transition of care decision intervention priority score, related data, and/or information. The user interface 400b includes an interactive element to navigate back to the user interface 400a (e.g., shown as an icon displaying a left pointing chevron within a circle) as well as an interactive element for system settings or configuration details (e.g., shown as three vertical dots, which is identical to system settings element 402 described in relation to FIG. 4A). The user interface 400b also includes patient data filters 414, patient identification data 416, patient priority indicator 418, and patient priority score data 420.

As shown in FIG. 4B, the user interface 400b provides healthcare practitioners with a graphical display identifying a list of patients who have been assigned to a particular priority category based on the patient's transition of care decision intervention priority score, related data, and/or information. For example, the user interface 400b is displaying a list of patients who have been assigned to the highest priority category. In some implementations, the user interface 400b may provide healthcare practitioners with a graphical display identifying a list patients of all patient priority categories.

As further shown in FIG. 4B, the user interface 400b includes patient data filters 414. The patient data filters 414 enable a healthcare practitioner to filter a list of patients who have been assigned to the highest priority category or a list of patients of all patient priority categories based on additional predetermined thresholds or filter criteria. As shown as an example in user interface 400b, the healthcare practitioner has selected to apply a filter to the list of all highest priority patients such that the user interface displays only the highest priority patients for whom a transition of care decision is due in less than or in 1 day. Upon executing the specific filter command that the healthcare practitioner has selected, the user interface 400b will display the list of patients for whom a transition of care decision is urgently due in less than or in 1 day. The patient data filter 414 may include a variety of other pre-configured or user-defined filter selection settings corresponding to a range of possible transition of care decision deadlines into the future. In some implementations, the patient data filter 414 may include other filter selection settings, including but not limited to, filtering patients by characteristics of the hospitalization, length of stay (LOS) in the hospital, age, acuity of diagnosis, comorbidities, and frailty risk. In some implementations, the patient data filter 414 may include a filter selection setting for applying no filter.

As further shown in FIG. 4B, the user interface 400b includes patient identification data 416. The patient identification data 416 includes personal and administrative data for use by healthcare practitioners for determining the identity and location of a particular patient. For example, the patient identification data 416 may include, but is not limited to, the patient's name, patient's medical record number, the hospital ward in which the patient is being treated, and the bed number that the patient is occupying in the hospital ward. A wide variety of other personal and administrative data could also be presented as patient identification data 416. In some implementations, the display of the specific patient identification data 416 may be user-defined or may be pre-configured.

As further shown in FIG. 4B, the user interface 400b includes patient priority indicator 418. In some implementations, where the user interface 400b is not displaying a list of patients who have been assigned to a particular priority category, for example, the highest priority category, but is instead displaying a list of patients of all patient priority categories or a list of patients requiring time-critical or prioritized transition of care decision interventions, the patient priority indicator 418 identifies the priority category for each patient in the list. The patient priority indicator 418 may include symbols (such as a shape, a regular or flashing exclamation point, a colored icon, or a combination of any of these) that alert a healthcare practitioner of the patients' priority category. The indicators described herein are mere examples and any other suitable indicators, symbols, or alert systems that are capable of conveying the priority categories may be employed for this purpose. For example, the patient priority indicator 418 may include a grey square with a white dash indicating no priority, a purple square with an exclamation point indicating low priority, a green square with an exclamation point indicating medium priority, a yellow triangle with an exclamation point indicating high priority, or a red circle with a regular or flashing exclamation point indicating highest priority category. For example, as shown in user interface 400b, the patient priority indicator 418 for every patient displays a red circle with an exclamation point indicating the highest priority category since the user interface 400b is set to display a list of patients who have been assigned to the highest priority category only.

As further shown in FIG. 4B, the user interface 400b includes patient priority score data 420. The patient priority score data 420 identifies a variety of information corresponding to the transition of care decision intervention. As shown in user interface 400b, the patient priority score data 420 for each patient includes an icon displaying a right pointing chevron within a circle, which when selected transitions to or displays a new user interface where the variety of information corresponding to the transition of care decision intervention of an individual patient is displayed. The user interface displaying the variety of information corresponding to the transition of care decision intervention for an individual patient will be described in relation to FIG. 4C.

FIG. 4C illustrates an example user interface 400c on a computing device displaying a variety of information corresponding to the transition of care decision intervention for an individual patient. Healthcare practitioners may interact with user interface 400c to review a patient's transition of care decision intervention priority score and a variety of information corresponding to the patient's transition of care decision intervention and priority indicator, review the patient's current condition, review the patient's treatment notes, and to enter treatment instructions for the transition of care decision intervention for the patient. The user interface 400c includes an interactive element 422 to navigate back to user interfaces 400a or 400b as well as a patient priority indicator 424 similar to the patient priority indicator 416 shown in FIG. 4B and interactive elements for system settings or configuration details (e.g., shown as three vertical dots). The user interface 400c also includes patient identification data 426, current encounter summary 428, an overview element 430, a review notes element 432, an enter instructions element 434, patient's discharge planning insights data 436, patient's recommended providers data 438, and a patient's utilization history data 440.

As shown in FIG. 4C, the user interface 400c includes patient identification data 426. The patient identification data 426 is similar to the patient identification data 420 shown in FIG. 4B. User interface 400c may include additional or fewer patient identification data elements as required to accurately identify individual patients in the context displaying and interacting with transition of care decision intervention for an individual patient. For example, as shown in user interface 400c, the identification data 426 displays chronic condition for the patient J. Smith.

As further shown in FIG. 4C, the user interface 400c includes current encounter summary 428. The current encounter summary 428 provides a brief summary of the patient's current diagnoses, treatment planned, and/or treatment completed. For example, as shown in user interface 400c, the current encounter summary 428 displays patient J. Smith's current treatment completed as major hip and knee joint replacement or reattachment of lower extremity.

As further shown in FIG. 4C, the user interface 400c includes an overview element 430. The overview element 430 is an element in the user interface 400c that, when selected, displays a brief overview of reasons underlying the determined transition of care decision intervention or transition of care decision intervention priority score for a patient. The overview element 430, when selected, may further display recommendations for an optimal service of health care, care provider, and/or site of care for the patient and reasons for the same based on the transition of care decision intervention priority score for the patient. For example, the overview element 430 as shown in the user interface 400c, when selected may display the following overview for patient J. Smith:
Patient is a potential candidate for home discharge because: Advanced age and moderate acuity diagnosis make this a borderline case. However, this is the patient's first known admission in over a year. Considering low frailty risk and lack of significant comorbidities, this patient may be a potential candidate for home discharge.

As further shown in FIG. 4C, the user interface 400c includes a review notes element 432. The review notes element 432 is a graphical element in the user interface 400c that, when selected, displays the patient's medical charts, treatment notes, and/or any other configured data that has been linked to the review notes element to enable healthcare practitioners to view additional data pertaining to the patient's treatment in the hospital and transition of care decision intervention. The review notes element 432 enables a healthcare practitioner to view any patient data associated with the determination of the transition of care decision intervention priority scores for a patient. In some implementations, the review notes element 432 may further enable a healthcare practitioner to view automated clinical justifications for comorbidities, type, timing, nature, and degree of transition of care decision intervention for a patient. In other implementations, the review notes element 432 may further enable a healthcare practitioner to view clinical justifications for prioritization of one patient over another consistent with the urgency of the transition of care decision interventions at a given time, for example, in a list of patients assigned to the highest priority category as shown as an example in the user interface 400b in FIG. 4B.

As further shown in FIG. 4C, the user interface 400c includes an enter instructions element 434. The enter instructions element 434 is a graphical element in the user interface 400c that, when selected, displays an interface for the healthcare practitioner to enter instructions about the patient's treatment, care, transition of care decision intervention, discharge decision, and/or any other healthcare related instructions. In some implementations, upon reviewing a patient's data associated with the determination of the transition of care decision intervention priority scores displayed on the user interface 400c, the healthcare practitioner may take an action consistent with the transition of care decision intervention priority score by selecting the enter instructions element 434 and entering the discharge decision and instructions. The healthcare practitioner may select the enter instructions element 434 in the user interface 400c to enter discharge decision regarding optimal services of health care, care providers, and/or site of care for the patient, including for example, discharge from emergency department (ED) to inpatient hospitalization, discharge from inpatient hospitalization to post-acute care facilities or services, discharge from inpatient hospitalization to hospice care, discharge from post-acute care to outpatient services, discharge from post-acute care to home or home care, and discharge from intensive care unit (ICU) to inpatient care. In some implementations, the healthcare practitioner may also select the enter instructions element 434 in the user interface 400c to enter decisions regarding certain patient segments (including Medicaid) and any other clinical decision spaces more broadly involving transition of care. In some implementations, by selecting the enter instructions element 434, the healthcare practitioner may be able to enter instructions and/or recommendations for follow-up and assessments for a particular patient. In other implementations, upon reviewing the patient's data associated with determination of the transition of care decision intervention priority scores displayed on the user interface 400c, the healthcare practitioner may manually override the patient's priority category auto-calculated based on the transition of care decision intervention priority score and assign the patient to a different priority category and/or to no priority by updating the patient priority indicator 424. In other implementations, the upon reviewing the patient's data associated with determination of the transition of care decision intervention priority scores displayed on the user interface 400c, the healthcare practitioner may take an action and/or enter a discharge decision regarding the optimal services of health care, care providers, and/or site of care that is different from the optimal services of health care, care providers, and/or site of care displayed in the overview element 430 of user interface 400c. The healthcare practitioner instructions described herein for user interface 400c are mere examples, and any other healthcare instructions related to a patient's treatment, care, transition of care decision intervention, and/or discharge decision may be entered by selecting the enter instructions element 434.

As further shown in FIG. 4C, the user interface 400c includes a discharge planning insights data 436. The discharge planning insights data 436 displays a list of transition of care discharge decision planning insights for a particular patient. In some implementations, the discharge planning insights data 436 for the patient may be based on available medical data for that patient for a given period of time. In some implementations, the discharge planning insights data 436 may display information on the patient's diagnoses, procedures, and comorbidities, indicators of socio-behavioral need, markers of frailty and decreased mobility, episodes of hospitalization, emergency department visits, outpatient visits, and previous post-acute care provider utilization history, for example, at Home Health Agencies (HHA), Skilled Nursing Facilities (SNF), Inpatient Rehabilitation Facilities (IRF), Long-Term Acute Care hospitals (LTACHs), etc. In some implementations, discharge planning insights may also include key markers of outcomes including hospital readmission rates and readmission risk. The discharge planning insights data described herein are mere examples and any other information useful for the transition of care discharge decision planning insights may be displayed in the discharge planning insights data 436. For example, in the user interface 400c, the discharge planning insights data 436 for patient J. Smith displays the following information:
Available data suggests that this is the first hospitalization for this patient.
Patient has not visited the ED in the past 12 months.
Patient's comorbidities are unlikely to prevent home discharge.

As further shown in FIG. 4C, the user interface 400c includes a recommended providers data 438. In some implementations, the recommended providers data 438 displays a shortlist of best-matched health care providers and/or health care facilities cross-checked with a particular patient's medical insurance. The recommended providers data 438 may display a shortlist of recommended health care providers and/or health care facilities based on other parameters related to a particular patient's social, financial, and any other relevant healthcare-related needs. For example, in the user interface 400c, the recommended providers data 438 for patient J. Smith displays a shortlist of best-matched HHAs and SNFs generated after cross-checking with the patient's medical insurance. In some implementations, the recommended providers data 438 may identify health care providers and/or facilities with which the hospital or healthcare system has a preferred relationship. The recommended providers data 438 may include an identifier, a symbol, or a text displayed next to the preferred provider and/or facility name.

As further shown in FIG. 4C, the user interface 400c includes a utilization history data 440. The utilization history data 440 displays the health care utilization history (e.g., at a health care facility and/or a health care system) for a particular patient. In some implementations, the utilization history data 440 may be filtered based on the provider type utilized by a particular patient, for example, hospitals, Skilled Nursing Facilities (SNF), Home Health Agencies (HHA), emergency departments (ED), and/or primary care facilities. In some implementations, the utilization history data 440 may further include, for a particular patient, information related to the name of the admitting provider or primary care physician (PCP), diagnosis-related group (DRG), admission date, discharge date, Length of Stay (LOS), diagnoses, procedures, comorbidities, discharge notes, and/or recovery history. The utilization history data described herein are mere examples and any other information related to a health care facility and/or health care system utilization history may be displayed in the utilization history data 440. For example, in the user interface 400c, the utilization history data 440 for patient J. Smith displays that the utilization history data has been filtered to display utilization history from hospitals, SNF, HHA, ED, and primary care, and for the one utilization event displayed, the patient was discharged to “home without skilled services.”

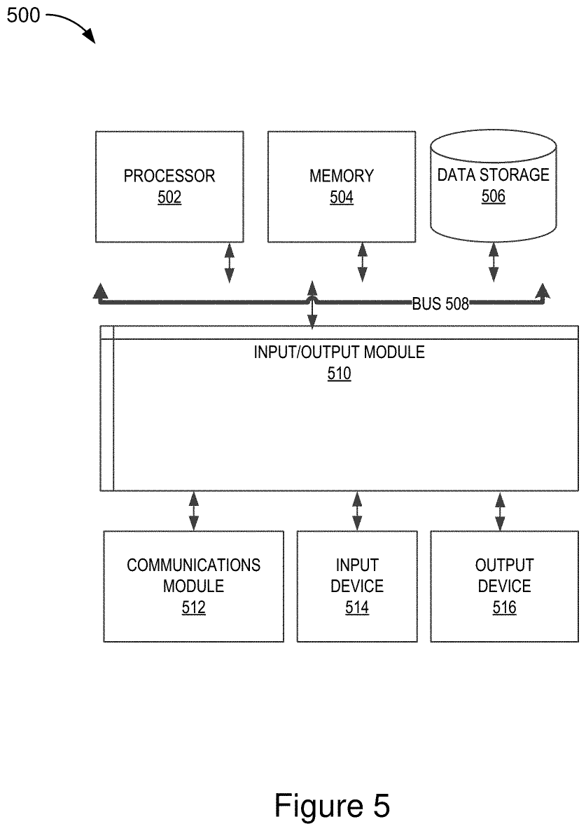

FIG. 5 is a block diagram illustrating an example computer system 500 with which the client 204 and servers 216, 228, 236, 250, 266, 270, and 274 of FIGS. 2A-2E can be implemented. In certain aspects, the computer system 500 may be implemented using hardware or a combination of software and hardware, either in a dedicated server, or integrated into another entity, or distributed across multiple entities.

Computer system 500 (e.g., client 204 and the servers disclosed herein) includes a bus 508 or other communication mechanism for communicating information, and a processor 502 (e.g., processors 206 and 220) coupled with bus 508 for processing information. According to one aspect, the computer system 500 can be a cloud computing server of an IaaS that is able to support PaaS and SaaS services. According to one aspect, the computer system 500 is implemented as one or more special-purpose computing devices. The special-purpose computing device may be hard-wired to perform the disclosed techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be large-format computer systems, portable computer systems, handheld devices, networking devices or any other device that incorporates hard-wired and/or program logic to implement the techniques. By way of example, the computer system 500 may be implemented with one or more processors 502. Processor 502 may be a general-purpose microprocessor, a microcontroller, a Digital Signal Processor (DSP), an ASIC, a FPGA, a Programmable Logic Device (PLD), a controller, a state machine, gated logic, discrete hardware components, or any other suitable entity that can perform calculations or other manipulations of information.

Computer system 500 can include, in addition to hardware, code that creates an execution environment for the computer program in question, e.g., code that constitutes processor firmware, a protocol stack, a database management system, an operating system, or a combination of one or more of them stored in an included memory (e.g., memory 208 or 222), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 508 for storing information and instructions to be executed by processors 208 or 220. The processor 502 and the memory 504 can be supplemented by, or incorporated in, special purpose logic circuitry. Expansion memory may also be provided and connected to computer system 500 through input/output module 510, which may include, for example, a SIMM (Single In-Line Memory Module) card interface. Such expansion memory may provide extra storage space for computer system 500, or may also store applications or other information for computer system 500. Specifically, expansion memory may include instructions to carry out or supplement the processes described above, and may include secure information also. Thus, for example, expansion memory may be provided as a security module for computer system 500, and may be programmed with instructions that permit secure use of computer system 500. In addition, secure applications may be provided via the SIMM cards, along with additional information, such as placing identifying information on the SIMM card in a non-hackable manner.

The instructions may be stored in the memory 504 and implemented in one or more computer program products, e.g., one or more modules of computer program instructions encoded on a computer readable medium for execution by, or to control the operation of, the computer system 500 and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multi-paradigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, embeddable languages, and xml-based languages. Memory 504 may also be used for storing temporary variable or other intermediate information during execution of instructions to be executed by processor 502.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 500 further includes a data storage device 506 such as a magnetic disk or optical disk, coupled to bus 508 for storing information and instructions. Computer system 500 may be coupled via input/output module 510 to various devices (e.g., device 514 or device 516. The input/output module 510 can be any input/output module. Example input/output modules 510 include data ports such as USB ports. In addition, input/output module 510 may be provided in communication with processor 502, so as to enable near area communication of computer system 500 with other devices. The input/output module 502 may provide, for example, for wired communication in some implementations, or for wireless communication in other implementations, and multiple interfaces may also be used. The input/output module 510 is configured to connect to a communications module 512. Example communications modules (e.g., communications module 512 include networking interface cards, such as Ethernet cards and modems).

The components of the system can be interconnected by any form or medium of digital data communication, e.g., a communication network. The communication network (e.g., communication network 214) can include, for example, any one or more of a personal area network (PAN), a local area network (LAN), a campus area network (CAN), a metropolitan area network (MAN), a wide area network (WAN), a broadband network (BBN), the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

For example, in certain aspects, communications module 512 can provide a two-way data communication coupling to a network link that is connected to a local network. Wireless links and wireless communication may also be implemented. Wireless communication may be provided under various modes or protocols, such as GSM (Global System for Mobile Communications), Short Message Service (SMS), Enhanced Messaging Service (EMS), or Multimedia Messaging Service (MMS), CDMA (Code Division Multiple Access), Time division multiple access (TDMA), Personal Digital Cellular (PDC), Wideband CDMA, General Packet Radio Service (GPRS), or LTE (Long-Term Evolution), among others. Such communication may occur, for example, through a radio-frequency transceiver. In addition, short-range communication may occur, such as using a BLUETOOTH, WI-FI, or other such transceiver.

In any such implementation, communications module 512 sends and receives electrical, electromagnetic or optical signals that carry digital data streams representing various types of information. The network link typically provides data communication through one or more networks to other data devices. For example, the network link of the communications module 512 may provide a connection through local network to a host computer or to data equipment operated by an Internet Service Provider (ISP). The ISP in turn provides data communication services through the world wide packet data communication network now commonly referred to as the “Internet”. The local network and Internet both use electrical, electromagnetic or optical signals that carry digital data streams. The signals through the various networks and the signals on the network link and through communications module 512, which carry the digital data to and from computer system 500, are example forms of transmission media.

Computer system 500 can send messages and receive data, including program code, through the network(s), the network link and communications module 512. In the Internet example, a server might transmit a requested code for an application program through Internet, the ISP, the local network and communications module 512. The received code may be executed by processor 502 as it is received, and/or stored in data storage 506 for later execution.

In certain aspects, the input/output module 510 is configured to connect to a plurality of devices, such as an input device 514 (e.g., input device 201) and/or an output device 516 (e.g., output device 202). Example input devices 514 include a keyboard and a pointing device, e.g., a mouse or a trackball, by which a user can provide input to the computer system 500. Other kinds of input devices 514 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Example output devices 516 include display devices, such as a LED (light emitting diode), CRT (cathode ray tube), LCD (liquid crystal display) screen, a TFT LCD (Thin-Film-Transistor Liquid Crystal Display) or an OLED (Organic Light Emitting Diode) display, for displaying information to the user. The output device 516 may comprise appropriate circuitry for driving the output device 516 to present graphical and other information to a user.

According to one aspect of the present disclosure, the client 204 and servers 216, 228, 236, 250, 266, 270, and 274 of FIGS. 2A-2E can be implemented using a computer system 500 in response to processor 502 executing one or more sequences of one or more instructions contained in memory 504. Such instructions may be read into memory 504 from another machine-readable medium, such as data storage device 506. Execution of the sequences of instructions contained in main memory 504 causes processor 502 to perform the process steps described herein. One or more processors in a multi-processing arrangement may also be employed to execute the sequences of instructions contained in memory 504. Processor 502 may process the executable instructions and/or data structures by remotely accessing the computer program product, for example by downloading the executable instructions and/or data structures from a remote server through communications module 512 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

Computing system 500 can include clients and servers. A client and server are generally remote from each other and typically interact through a communication network. The relationship of client and server arises by virtue of computer programs running on the respective computers and having a client-server relationship to each other. Computer system 500 can be, for example, and without limitation, a desktop computer, laptop computer, or tablet computer. Computer system 500 can also be embedded in another device, for example, and without limitation, a mobile telephone, a personal digital assistant (PDA), a mobile audio player, a Global Positioning System (GPS) receiver, a video game console, and/or a television set top box.

The term “machine-readable storage medium” or “computer-readable medium” as used herein refers to any medium or media that participates in providing instructions or data to processor 502 for execution. The term “storage medium” as used herein refers to any non-transitory media that store data and/or instructions that cause a machine to operate in a specific fashion. Such a medium may take many forms, including, but not limited to, non-volatile media, volatile media, and transmission media. Non-volatile media include, for example, optical disks, magnetic disks, or flash memory, such as data storage device 506. Volatile media include dynamic memory, such as memory 504. Transmission media include coaxial cables, copper wire, and fiber optics, including the wires that comprise bus 508. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 608. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in other one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted that the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A computer-implemented method for transition of care decision intervention using machine learning, the method comprising:
(a) receiving patient data, the patient data including values for a plurality of features associated with a first patient;
(b) determining a first transition of care decision score for the first patient by processing the patient data through a first, historical decision-derived transition of care decision model and at least a second transition of care decision score for the first patient by processing the patient data through at least a first, expert recommendation-derived transition of care decision model;
(c) calculating a first transition of care decision intervention priority score for the first patient based on a degree of difference between the first and second transition of care decision scores for the first patient; and
(d) displaying on a graphical interface, data corresponding to the first transition of care decision intervention priority score for the first patient.

2. The computer-implemented method of claim 1, wherein the intervention comprises at least one of revaluating or assigning additional resources to a health facility discharge decision, clinical triage decision, functional assessment, social needs assessment, and/or a care plan associated with the transition of care.

3. The computer-implemented method of claim 1, wherein the plurality of features comprises features from a majority of the following feature categories: patient demographic data, patient clinical data, patient financial data, administrative data including patient health insurance information and claims data, patient health care utilization history, patient prior recovery data, data indicative of patient's access to physicians and clinical caregivers, patient socio-economic data, and patient behavioral health data.

4. The computer-implemented method of claim 1, the method further comprising:
(a) receiving patient data including values for a plurality of features associated with at least one additional patient;
(b) determining transition of care decision scores for the at least one additional patient;
(c) calculating a transition of care decision intervention priority score for the at least one additional patient; and
(d) displaying on a graphical user interface, the data corresponding to transition of care decision intervention priority score for the at least one additional patient.

5. The computer-implemented method of claim 4, wherein the first patient and the at least one additional patient comprise a patient population, and wherein the data corresponding to the transition of care priority scores for the patients in the patient population is displayed on the graphical user interface, organized according to a ranking of the patients in the patient population according to their relative transition of care decision intervention priority scores.

6. The computer-implemented method of claim 5, wherein the patient population comprises the patient population of a health care facility.

7. The computer-implemented method of claim 1, the method further comprising:
(a) determining at least one additional transition of care decision score for the first patient by processing the patient data through at least one additional expert recommendation-derived transition of care decision model;
(b) calculating an aggregated value for an expert recommendation-derived transition of care decision score for the first patient by performing an aggregation function on the second transition of care decision score and the at least one additional transition of care decision score determined by processing the patient data through respective first and the at least one additional expert recommendation-derived transition of care decision models; and
(c) wherein calculating the first transition of care decision intervention priority score for the first patient is based on the degree of difference between the first transition of care decision score and the said aggregated value for the expert recommendation-derived transition of care decision score.

8. The computer-implemented method of claim 1, the method further comprising displaying on the graphical user interface at least one or more of the following information types:
(a) explanatory information underlying a transition of care decision intervention recommendation for a patient comprising at least one of:
(i) clinical justifications for a transition of care decision intervention,
(ii) indicators of socio-behavioral needs,
(iii) markers of frailty and decreased mobility, and
(iv) prior health care utilization and recovery history; and
(b) a personalized list of recommendations for a patient comprising at least one of:
(i) recommended health care services, care providers, facilities and agencies cross-checked with a patient's medical insurance,
(ii) recommendations for follow-up assessments,
(iii) recommendations for clinical interventions by future providers, and
(iv) a recommended duration for at least one or more of the following: a clinical intervention, institutionalization, series of home health care provider visits, and hospitalization.

9. A non-transitory computer-readable medium storing instructions that, when executed by a processor, cause the processor to perform a method for transition of care decision intervention using machine learning, the method comprising:
(a) receiving patient data, the patient data including values for a plurality of features associated with a first patient;
(b) determining a first transition of care decision score for the first patient by processing the patient data through a first, historical decision-derived transition of care decision model and at least a second transition of care decision score for the first patient by processing the patient data through at least a first, expert recommendation-derived transition of care decision model;
(c) calculating a first transition of care decision intervention priority score for the first patient based on a degree of difference between the first and second transition of care decision scores for the first patient; and
(d) displaying on a graphical interface, data corresponding to the first transition of care decision intervention priority score for the first patient.

10. The non-transitory computer-readable medium of claim 9, wherein the method further comprises:
(a) receiving patient data including values for a plurality of features associated with at least one additional patient;
(b) determining transition of care decision scores for the at least one additional patient;
(c) calculating a transition of care decision intervention priority score for the at least one additional patient; and
(d) displaying on a graphical user interface, the data corresponding to transition of care decision intervention priority score for the at least one additional patient; and
wherein the first patient and the at least one additional patient comprise a patient population, and wherein the data corresponding to the transition of care priority scores for the patients in the patient population is displayed on the graphical user interface, organized according to a ranking of the patients in the patient population according to their relative transition of care decision intervention priority scores.

11. The non-transitory computer-readable medium of claim 9, wherein the method further comprises displaying on the graphical user interface at least one or more of the following information types:
(a) explanatory information underlying a transition of care decision intervention recommendation for a patient comprising at least one of:
(i) clinical justifications for a transition of care decision intervention,
(ii) indicators of socio-behavioral needs,
(iii) markers of frailty and decreased mobility, and
(iv) prior health care utilization and recovery history; and
(b) a personalized list of recommendations for a patient comprising at least one of:
(i) recommended health care services, care providers, facilities and agencies cross-checked with a patient's medical insurance,
(ii) recommendations for follow-up assessments,
(iii) recommendations for clinical interventions by future providers, and
(iv) a recommended duration for at least one or more of the following: a clinical intervention, institutionalization, series of home health care provider visits, and hospitalization.

12. A system for transition of care decision intervention using machine learning, the system comprising:
a memory storing computer-readable instructions and a plurality of transition of care decision intervention models; and
a processor, the processor configured to execute the computer-readable instructions, which when executed carry out the method comprising:
(a) receiving patient data, the patient data including values for a plurality of features associated with a first patient;
(b) determining a first transition of care decision score for the first patient by processing the patient data through a first, historical decision-derived transition of care decision model and at least a second transition of care decision score for the first patient by processing the patient data through at least a first, expert recommendation-derived transition of care decision model;
(c) calculating a first transition of care decision intervention priority score for the first patient based on a degree of difference between the first and second transition of care decision scores for the first patient; and
(d) displaying on a graphical interface, data corresponding to the first transition of care decision intervention priority score for the first patient.

13. The system of claim 12, wherein the intervention comprises at least one of revaluating or assigning additional resources to a health facility discharge decision, clinical triage decision, functional assessment, social needs assessment, and/or a care plan associated with the transition of care.

14. The system of claim 12, wherein the plurality of features comprises features from a majority of the following feature categories: patient demographic data, patient clinical data, patient health insurance claims data, patient financial data, administrative data including patient health insurance information and claims data, patient health care utilization history, patient prior recovery data, data indicative of patient's access to physicians and clinical caregivers, patient socio-economic data, and patient behavioral health data.

15. The system of claim 12, wherein the memory further stores computer-readable instructions, which when executed cause the processor to carry out the method further comprising:
(a) receiving patient data including values for a plurality of features associated with at least one additional patient;
(b) determining transition of care decision scores for the at least one additional patient;
(c) calculating a transition of care decision intervention priority score for the at least one additional patient; and
(d) displaying on a graphical user interface, the data corresponding to transition of care decision intervention priority score for the at least one additional patient.

16. The system of claim 15, wherein the first patient and the at least one additional patient comprise a patient population, and wherein the data corresponding to the transition of care priority scores for the patients in the patient population is displayed on the graphical user interface, organized according to a ranking of the patients in the patient population according to their relative transition of care decision intervention priority scores.

17. The system of claim 16, wherein the patient population comprises the patient population of a health care facility.

18. The system of claim 12, wherein the memory further stores computer-readable instructions, which when executed cause the processor to carry out the method further comprising:
(a) determining at least one additional transition of care decision score for the first patient by processing the patient data through at least one additional expert recommendation-derived transition of care decision model;
(b) calculating an aggregated value for an expert recommendation-derived transition of care decision score for the first patient by performing an aggregation function on the second transition of care decision score and the at least one additional transition of care decision score determined by processing the patient data through respective first and the at least one additional expert recommendation-derived transition of care decision models; and
(c) wherein calculating the first transition of care decision intervention priority score for the first patient is based on the degree of difference between the first transition of care decision score and the said aggregated value for the expert recommendation-derived transition of care decision score.

19. The system of claim 12, wherein the memory further stores computer-readable instructions, which when executed cause the processor to carry out the method further comprising displaying on the graphical user interface at least one or more of the following information types:
(a) explanatory information underlying a transition of care decision intervention recommendation for a patient comprising at least one of:
(i) clinical justifications for a transition of care decision intervention,
(ii) indicators of socio-behavioral needs,
(iii) markers of frailty and decreased mobility, and
(iv) prior health care utilization and recovery history; and
(b) a personalized list of recommendations for a patient comprising at least one of:
(i) recommended health care services, care providers, facilities and agencies cross-checked with a patient's medical insurance,
(ii) recommendations for follow-up assessments,
(iii) recommendations for clinical interventions by future providers, and
(iv) a recommended duration for at least one or more of the following: a clinical intervention, institutionalization, series of home health care provider visits, and hospitalization.
